(19) United States
(12) Patent Application Publication
(10) Pub. No.: US 2025/0229052 A1
(43) Pub. Date: Jul. 17, 2025

(54) MULTIMODAL PAIN MANAGEMENT SYSTEMS AND METHODS

(71) Applicants: Evanesce Medical, Inc., Excelsior, MN (US); Mayo Foundation for Medical Education and Research, Rochester, MN (US)

(72) Inventors: Jeffrey PETERS, Excelsior, MN (US); Stephen BOLEA, Excelsior, MN (US); Christine HUNT, Jacksonville, FL (US); Steven R. CLENDENEN, Ponte Vedra Beach, FL (US); Robert J. SPINNER, Rochester, MN (US); Norman S. TURNER, Rochester, MN (US); Lawrence WALES, Maplewood, MN (US); Houssam FARRES, Excelsior, MN (US); Patrick Jan BOLDENOW, Bloomington, MN (US)

(73) Assignees: Evanesce Medical, Inc., Excelsior, MN (US); Mayo Foundation for Medical Education and Research, Rochester, MN (US)

(21) Appl. No.: 18/855,883

(22) PCT Filed: Apr. 24, 2023
(86) PCT No.: PCT/US2023/066133

§ 371 Date: Oct. 10, 2024

Related U.S. Application Data
(60) Provisional application No. 63/334,373, filed on Apr. 25, 2022.
(60) Provisional application No. 63/478,285, filed on Jan. 3, 2023.

(51) Int. Cl. A61M 19/00; A61M 5/14; A61M 5/145; A61M 39/02; A61N 1/05; A61N 1/36

(52) U.S. Cl. CPC A61M 19/00; A61M 5/1413; A61M 39/0208; A61N 1/0504; A61N 1/36071; A61M 2005/14506; A61M 2205/054; A61M 2205/8243

(57) ABSTRACT

According to aspects disclosed herein, an infusion lead assembly may include a housing including a needle receptacle, a housing lumen, a pin receptacle, and a housing conductive trace; a connector including an connector needle, an internal lumen, a metal pin, and a connector conductive trace, and an infusion lead body including an infusion lumen, an exit port, an internal wire, and a distal electrode. The infusion lead assembly may form an electrical path to transmit electrical signals across the connector conductive trace, the metal pin, the housing conductive trace, the internal wire, and the distal electrode. The infusion lead assembly may form a fluid path to transmit fluid across the internal lumen, the connector needle, the infusion lumen, and the exit port.

### CROSS-REFERENCE TO RELATED APPLICATIONS

This application claims the benefit of U.S. Provisional Patent Application 63/334,373, filed Apr. 25, 2022, and U.S. Provisional Patent Application 63/478,285, filed Jan. 3, 2023, the entire contents of each of which are incorporated herein by reference.

### FIELD

The present disclosure generally relates to devices, systems, and methods for pain management (e.g., motor pain management, sensory pain management, etc.) vasodilatation, and/or arteriovenous fistula (AVF) maturation.

### INTRODUCTION

Peripheral nerve blocks may be used to mitigate intra-operative and post-operative pain. A nerve block involves the injection of a local anesthetic around a nerve that innervates the surgical site. A drawback of nerve blocks is that they mitigate surgical pain for only a limited amount of time.

Opioids are often prescribed to address post-operative pain beyond the effective window of anesthetic injection. However, opioids are notoriously addictive, often leading to a potential cascade of social and health problems, including death.

This introduction section is provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the disclosure, methods and systems are disclosed for multimodal pain management.

According to aspects disclosed herein, an infusion lead assembly includes a housing including a needle receptacle, a housing lumen, a pin receptacle, and a housing conductive trace; a connector including an connector needle, an internal lumen, a metal pin, and a connector conductive trace; and an infusion lead body including an infusion lumen, an exit port, an internal wire, and a distal electrode, wherein the infusion lead assembly forms an electrical path to transmit electrical signals across the connector conductive trace, the metal pin, the housing conductive trace, the internal wire, and the distal electrode and wherein the infusion lead assembly forms a fluid path to transmit fluid across the internal lumen, the connector needle, the infusion lumen, and the exit port.

The housing may include a first gasket forming a fluid seal with the needle receptacle and a second gasket forming a fluid seal with the pin receptacle. The housing may include a first gasket forming a fluid seal with the needle receptacle and a second gasket forming a fluid seal with the pin receptacle and wherein at least one of the first gasket or the second gasket comprises an in-line filter. The housing may include a first gasket forming a fluid seal with the needle receptacle and a second gasket forming a fluid seal with the pin receptacle, wherein at least one of the first gasket or the second gasket comprises an in-line filter, and wherein the in-line filter includes an anti-bacterial material or an anti-bacterial coating. The housing may include a housing connection component and the connector may include a connector connection component, the housing connection component shaped to receive the connector connection component. The housing may include a housing connection component and the connector may include a connector connection component, the connector connection component shaped to receive the housing connection component. The housing lumen may be configured to provide fluid communication with a drug pump, the connector needle in connection with the drug pump via an infusion tube, the infusion lumen, and the exit port. The drug pump may include a spring system including at least one spring, where depressing the spring may trigger transmission of the fluid across the internal lumen and retraction of the spring may cause a second fluid to be retrieved into the drug pump from an external container. The pin receptacle may include an electrical surface for electrical communication with a pulse generator, the housing conductive trace, the internal wire, and the distal electrode. A proximal electrode may be included. An anchor may be included. The connector conductive trace may be in electrical communication with a pulse generator cable connected to a pulse generator. The housing may be configured to be placed subcutaneously and may further comprise: a subcutaneous injection port; and a sealing gasket sealingly attached to the subcutaneous injection port.

According to other aspects disclosed herein, an infusion lead assembly includes: a receiver including a receiver antenna to wirelessly receive power from a transmission component; a subcutaneous housing comprising a needle receptacle, a housing lumen, a pin receptacle, and a housing conductive trace; and an infusion lead body including an infusion lumen, an exit port, an internal wire, and a distal electrode, wherein the infusion lead assembly forms an electrical path to transmit electrical signals across the receiver antenna, the housing conductive trace, the internal wire, and the distal electrode and wherein the infusion lead assembly forms a fluid path to transmit fluid across the infusion lumen and the exit port.

The power may be transmitted by a transmission module. The subcutaneous housing may include a subcutaneous injection port in fluid communication with the infusion lumen.

According to other aspects disclosed herein, a method for multimodal stimulation may include receiving an electrical signal at a connector; transmitting the electrical signal through a connector conductive trace, a connector metal pin, a housing pin receptacle, a housing conductive trace, and an infusion lead body internal wire to an internal infusion lead body distal electrode at a first time; receiving a fluid at the connector; and transmitting the fluid through a connector internal lumen, a connector needle, a housing needle receptacle, a housing lumen, and an infusion lead body infusion lumen to an infusion lead body exit port at a second time.

The first time and the second time may be approximately a same time. The electrical signal or the fluid may be received based on a user input. At least one of the electrical signal or the fluid may be received based on a pre-programmed setting. The electrical signal may be received at a wireless subcutaneous receiver and wherein the fluid is received at a subcutaneous injection port. Transmitting the fluid through the exit port at the second time may cause at least one of pain management, vasodilation of a tissue, a vein, and/or nerve, or arteriovenous fistula (AVF) maturation.

The above summary is not intended to describe each and every embodiment or implementation of the present disclosure.

### BRIEF DESCRIPTION OF THE FIGURES

The accompanying drawings, which are incorporated into and constitute a part of this specification, illustrate various exemplary embodiments and, together with the description, serve to explain the principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

The drawings illustrate example embodiments of the present disclosure and, along with the description, serve to explain the principles of the disclosure. The drawings are only illustrative of certain embodiments and do not limit the disclosure or invention.

FIG. 1 is a schematic illustration showing a portion of a multimodal system, according to an example embodiment of the present disclosure.

FIG. 1A is a schematic illustration showing a drug pump, for use with the system of FIG. 1, according to an example embodiment of the present disclosure.

FIG. 1B is a schematic illustration showing a pulse generator, for use with the system of FIG. 1, according to an example embodiment of the present disclosure.

FIG. 1C is a schematic illustration showing a combined drug pump and pulse generator, for use with the system of FIG. 1, according to an example embodiment of the present disclosure.

FIG. 1D is a schematic illustration of another drug pump, according to an example embodiment of the present disclosure.

FIG. 1E is a schematic illustration of an internal view of the drug pump of FIG. 1D, according to an example embodiment of the present disclosure.

FIG. 1F is a flow diagram of operation of the drug pump of FIG. 1D, according to an example embodiment of the present disclosure.

FIG. 2 is a schematic illustration showing a housing, an adhesive patch, and a connector, according to an example embodiment of the present disclosure.

FIG. 2A is a schematic illustration showing another connector, according to an example embodiment of the present disclosure.

FIG. 2B is a schematic illustration showing another connector, according to an example embodiment of the present disclosure.

FIG. 2C is a schematic illustration showing a top view of the housing and adhesive patch of FIG. 2, according to an example embodiment of the present disclosure.

FIG. 2D is a schematic illustration showing a bottom view of the housing and adhesive patch of FIG. 2, according to an example embodiment of the present disclosure.

FIG. 2E is a schematic illustration showing a side view of the housing and adhesive patch of FIG. 2, according to an example embodiment of the present disclosure.

FIG. 2F is a schematic illustration showing a cap, according to an example embodiment of the present disclosure.

FIG. 2G is a schematic illustration showing another top view of the housing and adhesive patch of FIG. 2, according to an example embodiment of the present disclosure.

FIG. 2H is a schematic illustration of a drug pump, according to an example embodiment of the present disclosure.

FIG. 2I is a schematic illustration of another housing, according to an example embodiment of the present disclosure.

FIG. 2J is a schematic illustration of the interior of the housing of FIG. 2I, according to an example embodiment of the present disclosure.

FIG. 2K is a schematic illustration of a bottom portion of the housing of FIG. 2I, according to an example embodiment of the present disclosure.

FIG. 2L is a schematic illustration of a perspective view of the housing of FIG. 2I, according to an example embodiment of the present disclosure.

FIG. 2M is a schematic illustration of a wearable pump, according to an example embodiment of the present disclosure.

FIG. 2N is a schematic illustration of a back view of the wearable pump of FIG. 2M, according to an example embodiment of the present disclosure.

FIG. 2O is a schematic illustration of a wearable pump strap, according to an example embodiment of the present disclosure.

FIG. 2P is a schematic illustration of the wearable pump of FIG. 2M having a cover, according to an example embodiment of the present disclosure.

FIG. 2Q is a schematic illustration of another wearable pump, according to an example embodiment of the present disclosure.

FIG. 2R is a schematic illustration of a wearable pump attached to a user, according to an example embodiment of the present disclosure.

FIG. 2S is a schematic illustration of another wearable pump, according to an example embodiment of the present disclosure.

FIG. 2T is a schematic illustration of the wearable pump of FIG. 2S attached to a user, according to an example embodiment of the present disclosure.

FIG. 2U is a schematic illustration of another wearable pump, according to an example embodiment of the present disclosure.

FIG. 2V is a schematic illustration of the wearable pump of FIG. 2U attached to a user, according to an example embodiment of the present disclosure.

FIG. 2W is a schematic illustration of a needle guide system, according to an example embodiment of the present disclosure.

FIG. 2X is another schematic illustration of the needle guide system of FIG. 2W, according to an example embodiment of the present disclosure.

FIGS. 3A-3F are schematic illustrations for positioning an infusion lead body substantially parallel to a nerve, according to an example embodiment of the present disclosure.

FIG. 3G is a schematic illustration showing an infusion lead body inserted into a nerve sheath, according to an example embodiment of the present disclosure.

FIGS. 3H-3M are schematic illustrations for positioning an infusion lead body in a nerve sheath, according to an example embodiment of the present disclosure.

FIG. 4A is a schematic illustration showing an implantable pulse generator (IPG), according to an example embodiment of the present disclosure.

FIG. 4B is a schematic illustration showing a receiver (RX) module and a transmission (TX) module, according to an example embodiment of the present disclosure.

FIGS. 5A-5C are schematic illustrations showing another multimodal system, according to an example embodiment of the present disclosure.

FIG. 6 is a flowchart for bimodal, e.g., multimodal, stimulation, according to an example embodiment of the present disclosure.

FIG. 7 is a flowchart for infusion lead body placement, according to an example embodiment of the present disclosure.

FIG. 8A is a schematic illustration of an electronic placement detector, according to an example embodiment of the present disclosure.

FIG. 8B is a schematic illustration of a visual placement detector, according to an example embodiment of the present disclosure.

FIG. 9 is another schematic illustration of a multimodal system, according to an example embodiment of the present disclosure.

FIG. 10 is a flow diagram for training a machine learning model, according to an example embodiment of the present disclosure.

FIG. 11 is a schematic example of a computing device, according to an example embodiment of the present disclosure.

While embodiments of the disclosure are amenable to various modifications and alternative forms, specifics thereof have been shown by way of example in the drawings and will be described in some detail. It should be understood, however, that the intention is not to limit the disclosure to the particular embodiments described. On the contrary, the intention is to cover all modifications, equivalents, and alternatives falling within the spirit and scope of the invention.

### DETAILED DESCRIPTION

There are many embodiments described and illustrated herein. The described embodiments are neither limited to any single aspect nor implementation thereof, nor to any combinations and/or permutations of such aspects and/or implementations. Moreover, each of the aspects of the described embodiments, and/or implementations thereof, may be employed alone or in combination with one or more of the other aspects of the described embodiments and/or implementations thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

The term “distal end,” or any variation thereof, refers to the portion of a device farthest from an operator of the device during a procedure. Conversely, the term “proximal end,” or any variation thereof, refers to the portion of the device closest to the operator of the device. Further, any use of the terms “around,” “about,” “substantially,” and “approximately” generally mean+/−10% of the indicated value(s).

A way to address post-operative pain is the use a catheter and pump to deliver anesthetic over a prolonged period of time. However, the toxic nature of such anesthetics and/or the size and/or complexity of the catheters and pumps limits their standard use to several days post-operatively.

Peripheral nerve stimulation may be used to mitigate post-operative pain, but requires a separate procedure. The procedure involves inserting a lead with electrodes adjacent to the nerve that innervates the surgical site. A pulse generator is connected to the proximal end of the lead to deliver electrical stimulation to the nerve via the electrodes at the distal end of the lead. In a significant percentage of cases, nerve stimulation is not entirely effective at relieving post-operative pain. However, such cases are not identified until after the procedure is performed.

U.S. Pat. No. 7,386,350 to Vilims describes a combination electrical and chemical stimulation lead for use in intervertebral discs to promote tissue regeneration and repair.

U.S Patent Application Publication 2021/0330977 to Sinha describes a similar combination catheter, but for use in providing pain control. The catheter includes a lumen for the delivery of an anesthetic to the target nerve in addition to electrodes for the delivery of electrical stimulation to the target nerve. To avoid interference between the anesthetic solution and the electrical stimulation, Sinha proposes that the exit port(s) be spaced apart from the electrodes. However, this may be problematic if the port(s) and electrodes are at different distances from the nerve, because proximity influences power requirements, stray effects, concentration and dilution rates at the nerve, and ultimately efficacy.

Other shortcomings of such pain management systems in the prior art relate to practical challenges of self-administered pain management in the home setting, such as the use of an injection port requiring the patient to properly dose and inject anesthetic, the potential for migration of the stimulation electrodes, the potential for infection, etc. Further, the prior art does not address efficient conversion of post-operative (sub-chronic) pain management to chronic pain management, if needed.

Embodiments disclosed herein address an ongoing need to improve multimodal (chemical and electrical) pain management systems. For example, it is desirable to configure such systems and/or methods of use such that one mode of therapy does not compromise the other. It is also desirable to provide selective treatment (e.g., chemical and/or electrical stimulation) to provide motor pain management, sensory pain management, vasodilatation, and/or arteriovenous fistula (AVF) maturation. It is also desirable to configure such systems and/or methods such that they are suitable for use by patients in the home-setting without attendance by medical staff. Further, it is desirable to configure such systems such that they may be converted to partially or fully implantable systems to address chronic pain management if the need arises. The present disclosure offers a number of different embodiments to address these needs.

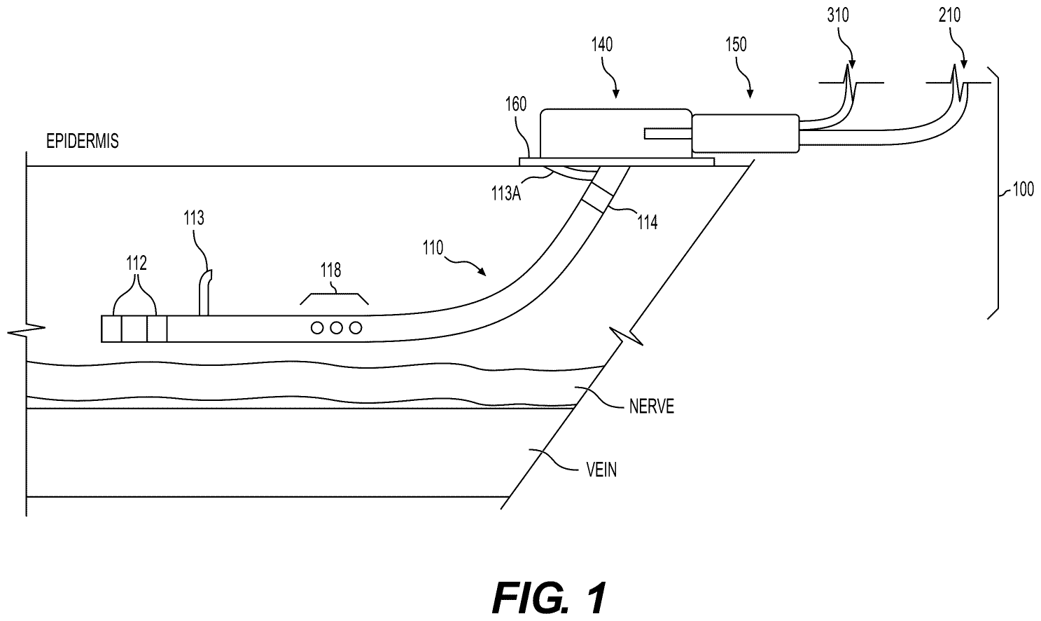

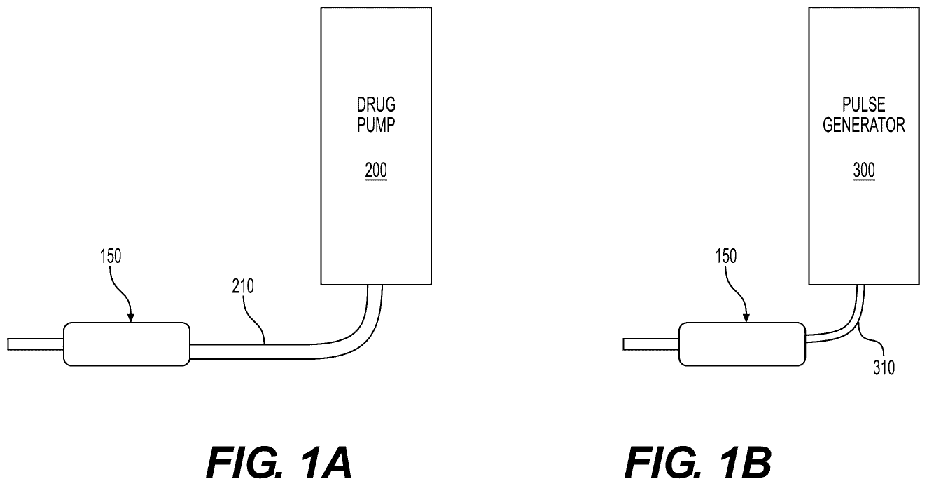

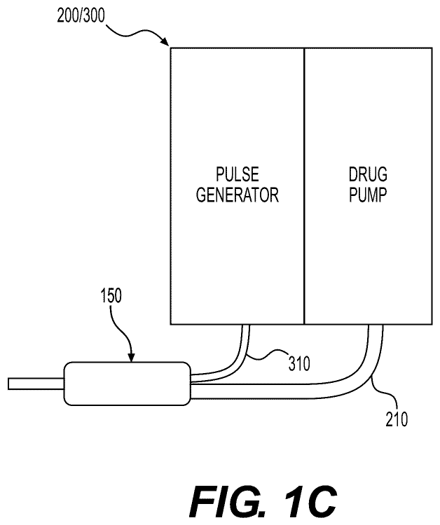

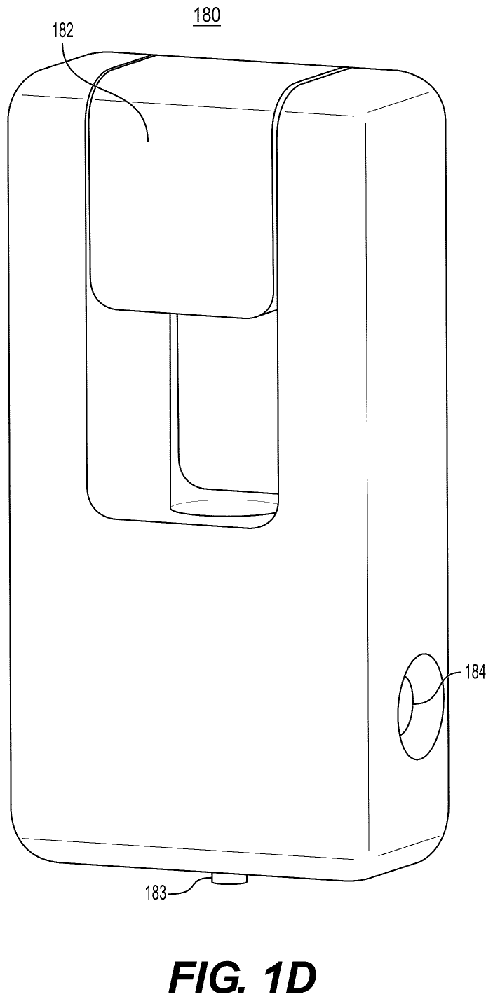

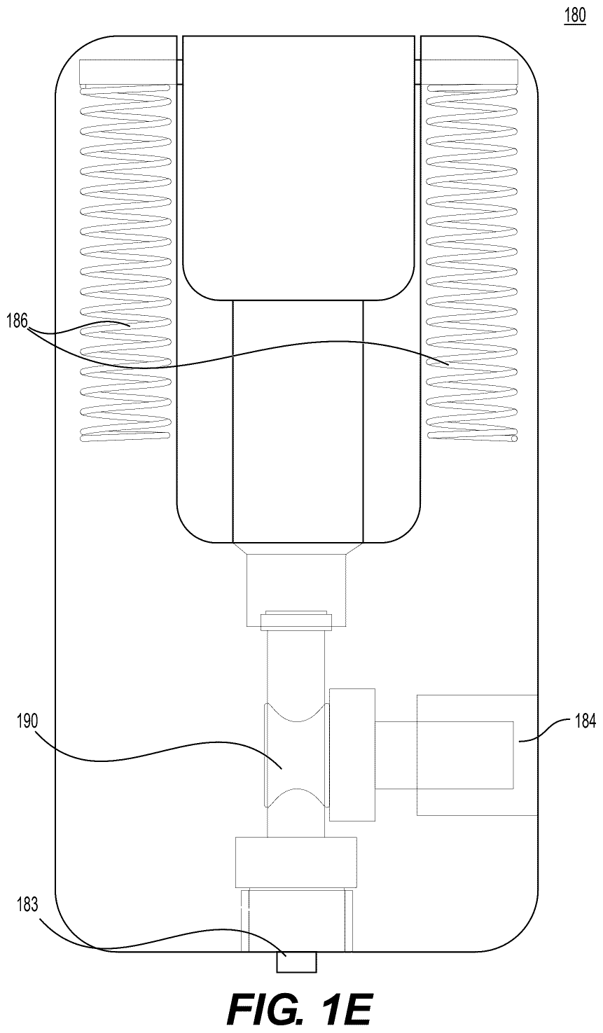

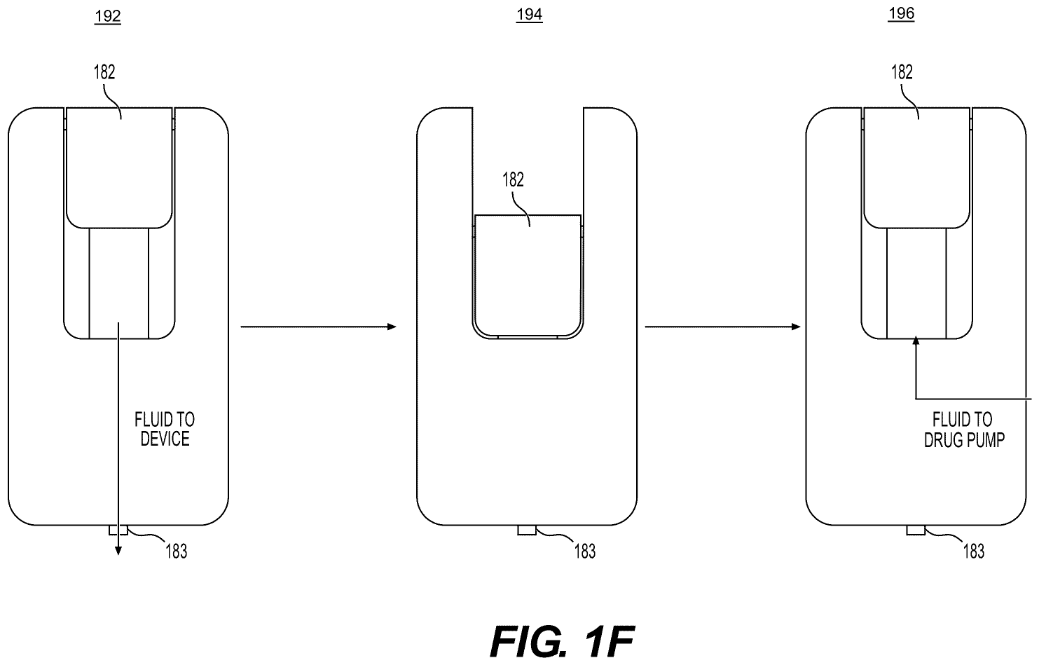

With reference to FIG. 1, a portion of a multimodal pain management system is shown schematically. The system may generally include an infusion lead assembly 100 configured for releasable connection to a drug pump 200 as shown in FIG. 1A, a pulse generator 300 as shown in FIG. 1B, or a combined drug pump and pulse generator 200/300 as shown in FIG. 1C via a housing 140, a connector 150 and associated infusion tube 210 and cable 310. The housing 140 may be secured to the epidermis via an adhesive patch 160 to mitigate migration. The top portion of the adhesive patch 160 may be attached (e.g., permanently) to the underside of the housing 140, and the bottom portion of the patch 160 may include an adhesive layer (e.g., suitable for approximately 10-14-day use under normal living conditions) covered by a removable covering (e.g., removable wax paper) until ready for application to the epidermis.

The infusion lead assembly 100 may include a tubular infusion lead body 110 having a proximal end connected to the housing 140. The infusion lead body 110 may include an infusion lumen (not visible) extending therethrough providing fluid communication between exit ports 118 and the drug pump 200 via housing 140, connector 150, and infusion tube 210 (e.g., when connector 150 is connected to housing 140). The infusion lead body 110 may further include one or more distal electrodes 112 and one or more proximal electrodes 114 in electrical communication with the pulse generator 300 via wires (not visible) embedded in the wall of the infusion lead body 110, via internal wires (not shown) extending through the housing 140 and connector 150, and via cable 310. The internal wires embedded in the wall of the infusion lead body 110 may extend alongside at least a portion of the infusion lumen of infusion lead body 110.

When inserted through and/or adhered to the skin as shown, the infusion lead body 110 may have a suitable length to position the distal electrodes 112 and exit ports 118 adjacent a nerve that innervates a surgical site. With this arrangement, a drug (e.g., anesthetic solution) may be delivered from the drug pump 200 to the nerve via ports 118, and electrical stimulation may be delivered from the pulse generator 300 to the nerve via electrodes 112, to provide combined chemical and electrical nerve block effects. Exit ports 118 may be a single opening or a plurality of openings.

Drug pump 200, pulse generator 300, connector 150, housing 140, and/or one or more other components disclosed herein may include or be associated with (e.g., in communication with) a safety mechanism. The safety mechanism may be configured to prevent chemical and/or electrical stimulation from being administered to a user in excess of a threshold property (e.g., prevent accidental stimulation). The safety mechanism may be implemented as a software component, hardware component, and/or firmware component. The safety mechanism may prevent or mitigate accidental stimulation and may be configured to be overridden by a user (e.g., a patient or an administrator). The threshold property may be, for example, a threshold amount (e.g., approximately 2cc, approximately 5cc, approximately 30v, etc.), a threshold time (e.g., approximately 5 seconds, approximately 20 seconds, etc.), a threshold frequency (e.g., within approximately 3 hours of a previous delivery, approximately once a day, etc.), and/or the like. For example, the safety mechanism may be a software or electronic component that tracks chemical and/or electrical properties (e.g., an amount, duration, time, etc.) of chemical and/or electrical stimulation. The safety mechanism may track one or more properties using one or more sensors such as a volume sensor (e.g., configured to track an amount of drug), a clock, a counter, a signal sensor, and/or the like. The safety mechanism may electronically prevent chemical and/or electrical stimulation (e.g., for a given duration of time) by transmitting a signal to a component (e.g., drug pump 200, pulse generator 300, a physical blocking component, etc.). As another example, the safety mechanism may be or may be associated with a physical component (e.g., a ticker, a physical counter, a lock, a valve, switch, etc.) configured to detect a chemical and/or electrical stimulation property and/or to prevent chemical and/or electrical stimulation (e.g., for a given amount of time, in excess of a given amount, etc.). The physical component may block or otherwise restrict chemical and/or electrical stimulation in response to a signal or in response to determining a chemical and/or electrical stimulation property reaching or exceeding a threshold value.

The electrical stimulation may be provided in a unipolar mode or a bipolar mode. For example, one of the distal electrodes 112 may serve as a cathode while the other distal electrode 112 serves as an anode. Alternatively, both distal electrodes 112 may be electrically shorted to serve as a combined anode or cathode, and the proximal electrode 114 may serve as a cathode or anode, respectively. The proximal electrode 114 may serve as effective ground.

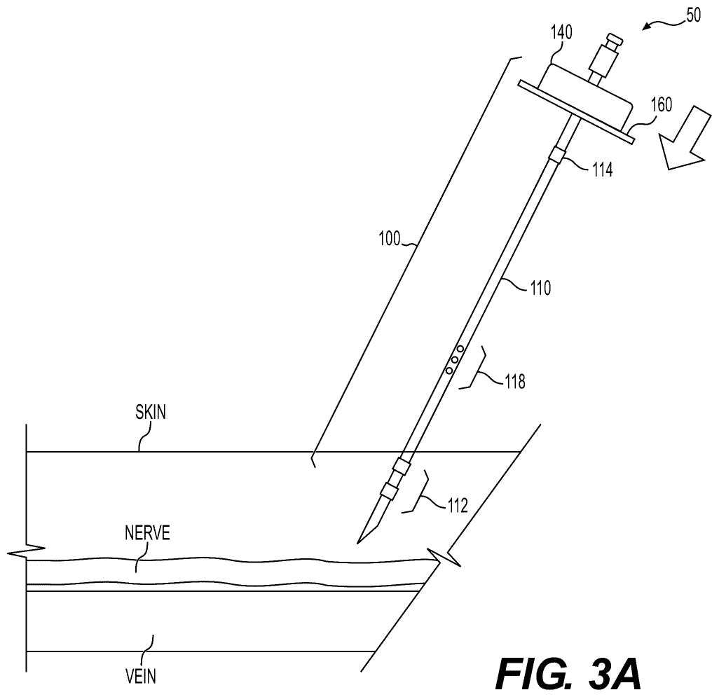

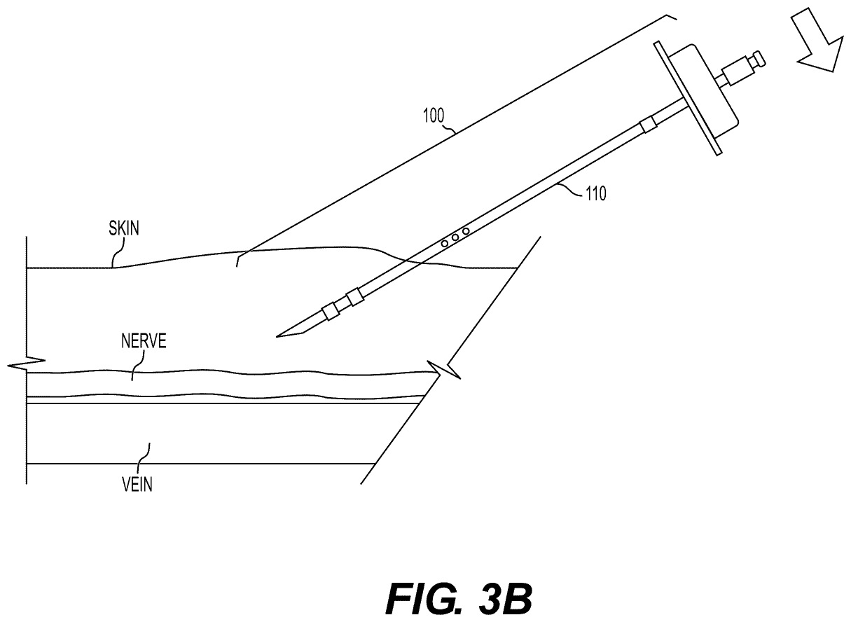

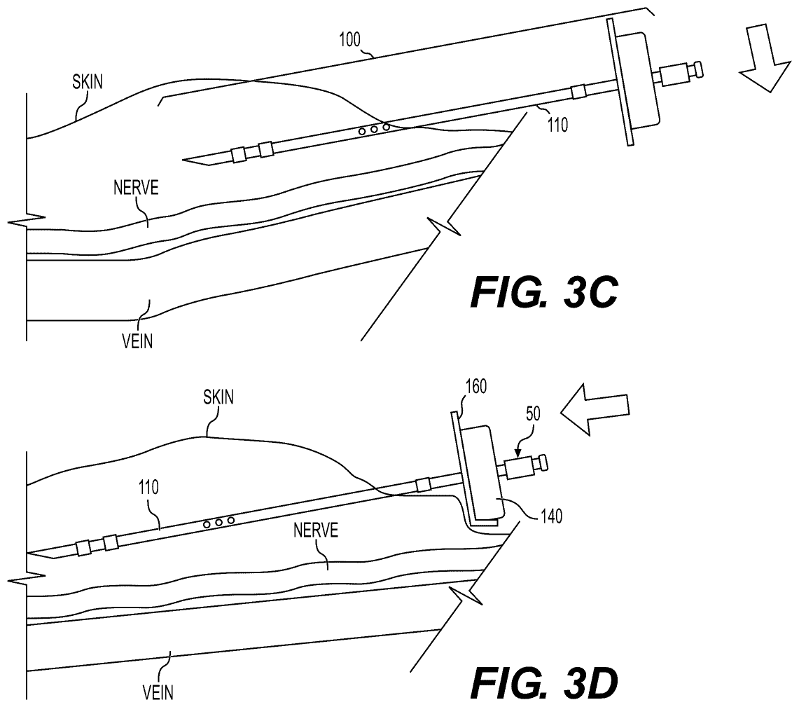

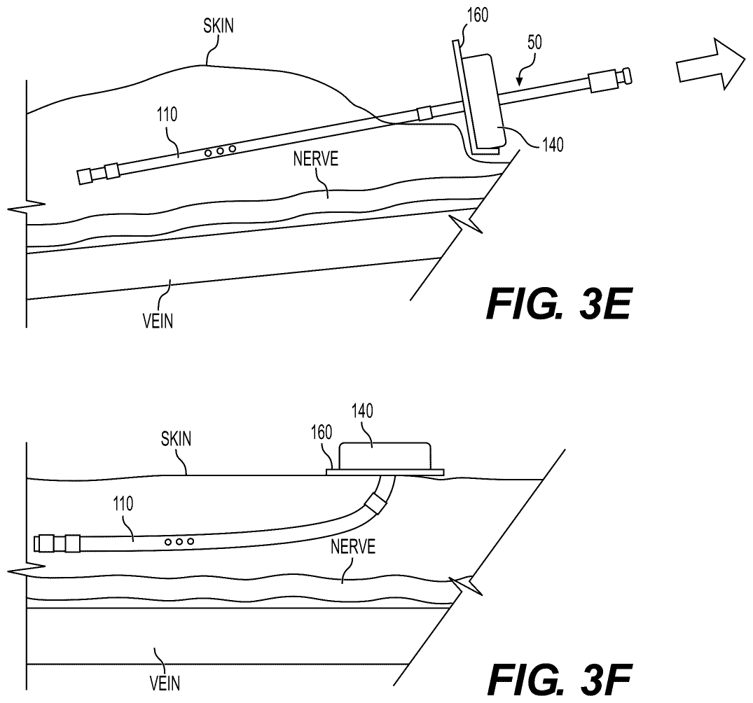

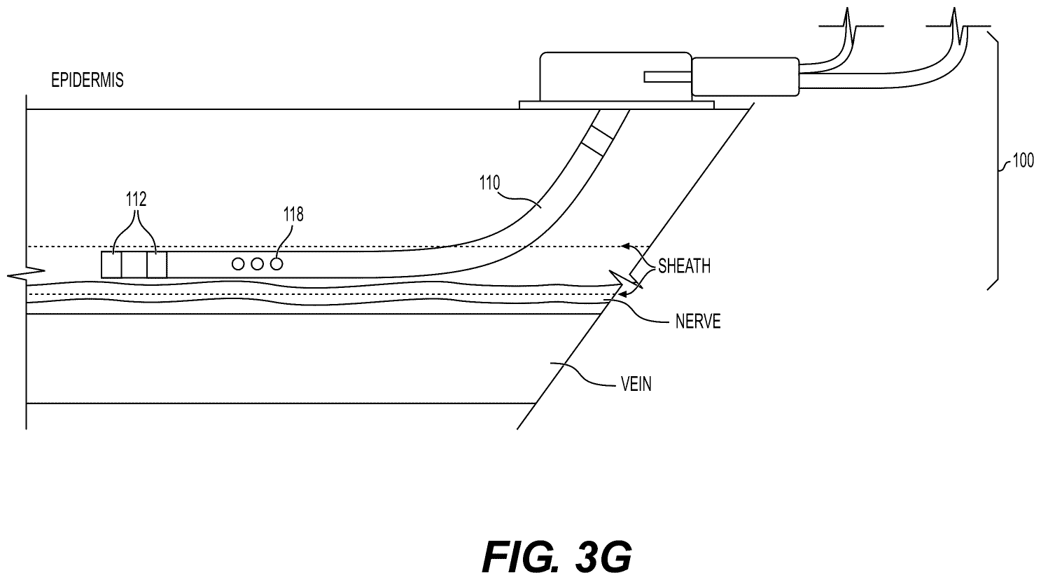

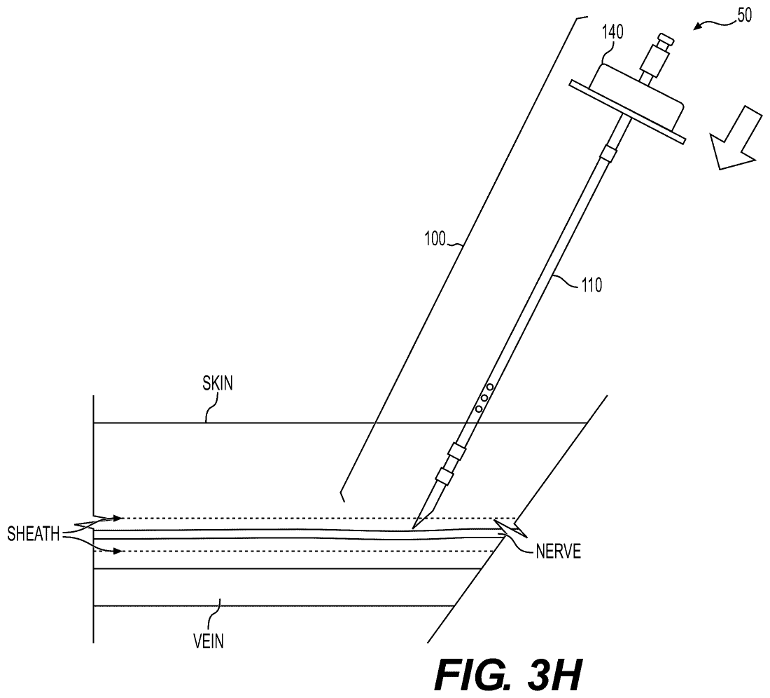

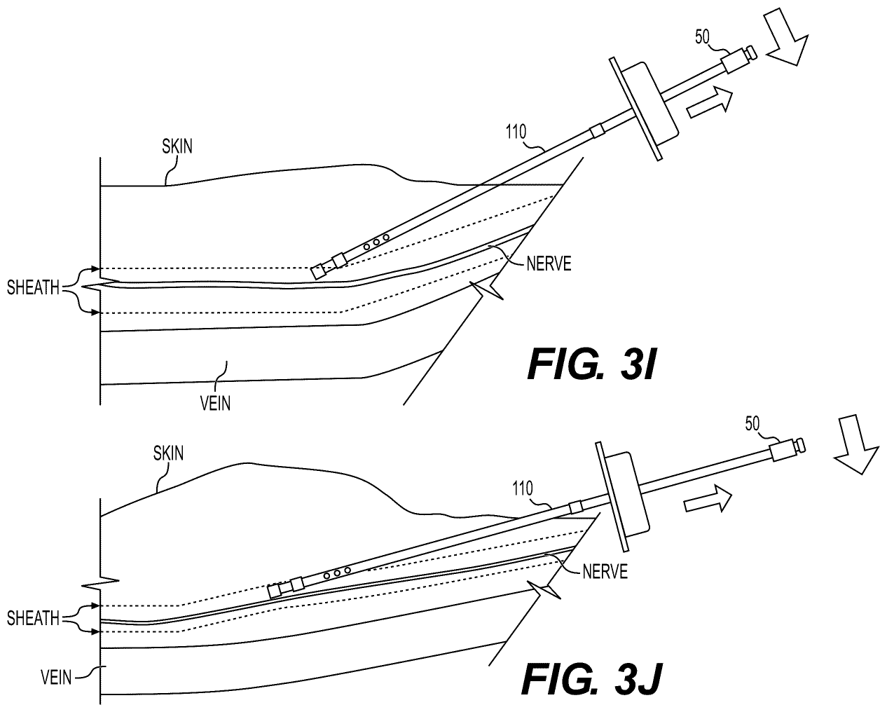

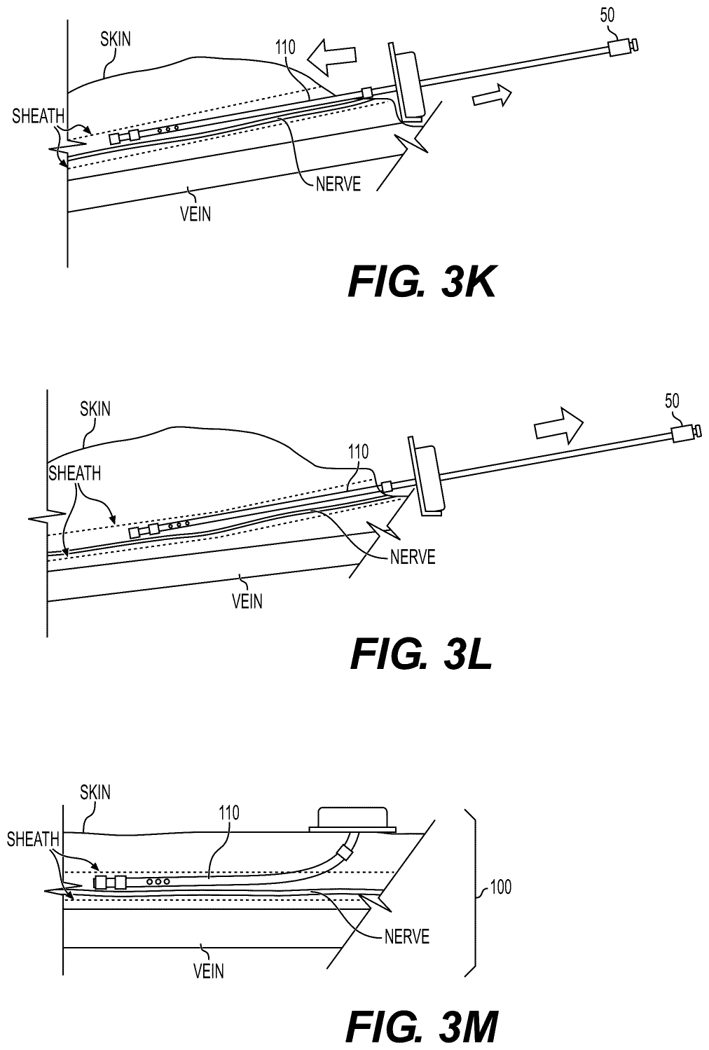

Infusion lead body 110 may further include or be attached to an anchor 113 or anchor 113A. Anchor 113 and/or anchor 113A may be shaped to or may include a material to secure infusion lead body 110 at a given location such that infusion lead body 110 remains proximate to and/or substantially parallel to a nerve. Anchor 113 and/or anchor 113A may be a cuff or other attachment mechanism. Anchor 113 and/or anchor 113A may prevent gross migration of infusion lead body 110 such that movement of lead body 110 is prevented or mitigated. Anchor 113 and/or anchor 113A may be configured to have strain relief (e.g., via coiling of anchor 113 and/or anchor 113A) such that the strain relief prevents or mitigates movement and/or detachment of anchor 113, anchor 113A, and or infusion lead body 110. Anchor 113 and/or anchor 113A may be include bioresorbable or biodegradable material, as discussed herein. Anchor 113 of infusion lead body 110 may attach to tissue (e.g., tissue proximate to a nerve) via any applicable attachment technique such as, but not limited to, a force connection, a friction connection, an adhesive connection, or the like or a combination thereof. For example, anchor 113 may include a proximal end attached to infusion lead body 110 and a distal end having a hook shape or C-shape. The hook shaped or C-shaped distal end may be latched to tissue such that the tissue is positioned within the hook or C-shape of the distal end of anchor 113. The distal end of anchor 113 may be latched to the tissue by rotating infusion lead body 110 and/or infusion lead assembly 100 during insertion of infusion lead body 110 and/or infusion lead assembly 100, as discussed in reference to FIGS. 3A-3F. Anchor 113A may be positioned proximate to the housing 140 and may attach to tissue proximate to housing 140, to a patient's skin (e.g., the underside of the patient's skin), and/or to adhesive patch 160. Anchor 113A may include a magnetic or metallic component which may magnetically attract a corresponding magnetic or metallic component of housing 140. Infusion lead body 110 may include no anchors, may include anchor 113 or anchor 113A, may include anchor 113 and anchor 113A, and/or any other applicable mechanism to secure infusion lead body 110 such that infusion lead body 110 remains proximate to and/or substantially parallel to a given nerve. Infusion lead body 110 may be braided to prevent coiling.

FIG. 1D is a schematic illustration of another drug pump 180, according to an example embodiment of the present disclosure. Drug pump 180 may be a spring-loaded drug pump and may be activated by depressing an activation button 182. Activation of drug pump 180 may cause a fluid (e.g., drug) stored at drug pump 180 to expel via port 183. Activation of drug pump 180 by depressing activation button 182 may trigger transmission of the fluid via port 183 and through an internal lumen (e.g., internal lumen 154, as further discussed herein) and/or to a delivery site (e.g., proximate to a nerve). Drug pump 180 may include an inlet 184 configured to retrieve a fluid from an external container (not shown), as further discussed herein in reference to FIG. 1E and FIG. 1F.

FIG. 1E is a schematic illustration of an internal view of drug pump 180. As shown, drug pump 180 may include a spring system including one or more resilient members, e.g., springs 186. Depressing activation button 182 may result in depressing springs 186 causing springs 186 to transition to a first position (e.g., loaded position). Depressing activation button 186 may cause the activation button 186 to lock in the depressed position for a given amount of time, until released by a user, and/or until released based on a signal. For example, a safety mechanism, as discussed herein, may generate a signal to release activation button 186 after a threshold amount of time has expired. The threshold amount of time may be determined by a machine learning model and/or based on user treatment plan. The locking of activation button 186 may mitigate or prevent accidental delivery of a drug in excess of an intended amount. After completion of a depressing action (e.g., releasing activation button 182, release of lock, signal from a safety mechanism, etc.), the loaded springs 186 expand from the first position to a second position (e.g., unloaded position). The change of springs 186 from the first position to the second position may cause the activation button 182 to expand to a first position (e.g., initial position) such that activation button 182 can be depressed a subsequent time, causing additional fluid to expel from port 183. Additionally, the change of springs 186 from the first position to the second position may cause a suction condition such that a suction pressure is applied via inlet 184. Inlet 184 may be connected to an external container that includes additional fluid (e.g., the same fluid expelled by drug pump 180 or a different fluid). The suction pressure may cause the additional fluid from the external container to be retrieved into drug pump 180, via inlet 184.

Drug pump 180 may include a valve system 190 (e.g., a dual valve system) that facilitates expelling fluid from drug pump 180, via port 183, upon depressing activation button 182. For example, a first valve component (not shown) of valve system 190 may be in an open position as fluid is expelled from drug pump 180, via port 183. The first valve component may transition to a closed position after the fluid is expelled and a second valve component (not shown) may transition from a closed position to an open position such that suction pressure is applied via inlet 184, as discussed herein. Accordingly, valve system 190 may facilitate expelling fluid via port 183 and may further facilitate retrieving additional fluid from an external container, via inlet 184. Valve system 190 and/or the second valve component may be configured such that no more than a given amount of fluid is retrieved from the external container. For example, valve system 190 and/or the second valve component may be configured such that only a given amount (e.g., approximately 2cc) of fluid is retrieved into drug pump 180, thereby preventing accidental delivery of a fluid in excess of the given amount of fluid.

FIG. 1F is a flow diagram illustrating example operation of drug pump 180. As shown at step 192, activation button 182 may be depressed from a first position to a second position, causing a fluid to be expelled from drug pump 180 to a device (e.g., connector 150), via port 183. Step 194 shows the activation button 182 in a second position, where springs 186 of FIG. 1E are in a first position (e.g., loaded position) and the fluid has been expelled via port 183. Springs 186 may transition from the first position to a second position (e.g., unloaded position), causing the activation button to return to the first position. The transition may cause application of the suction pressure discussed herein, resulting in additional fluid to be retrieved from an external container into drug pump 180, via inlet 184 of FIG. 1D and FIG. 1E.

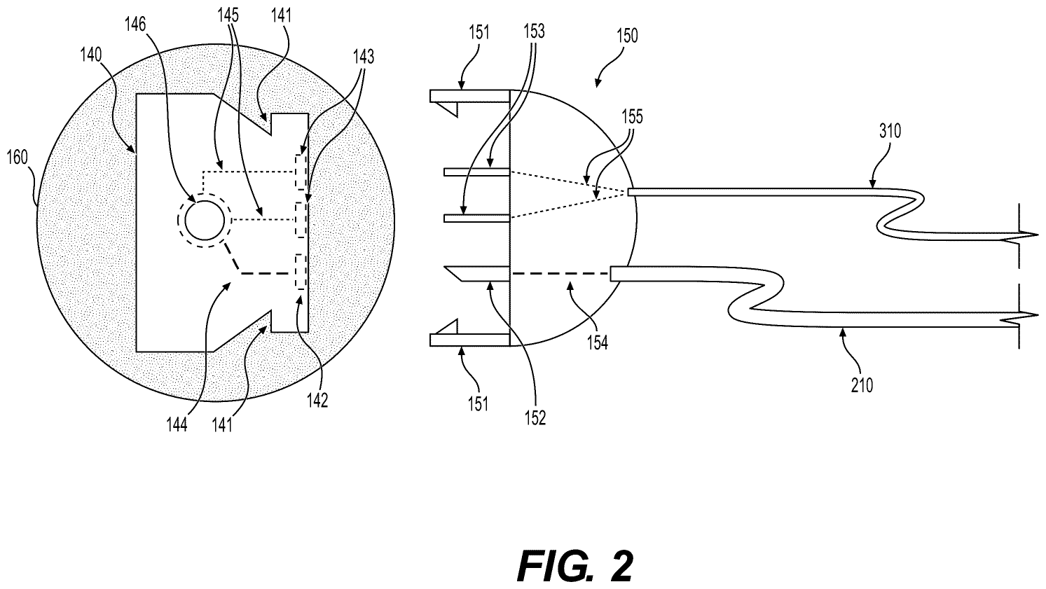

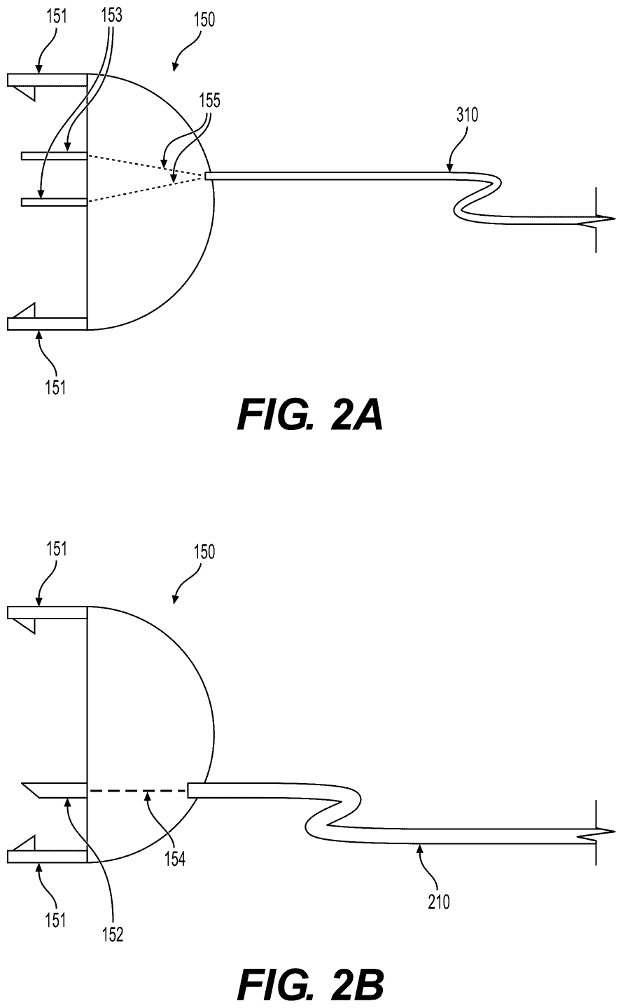

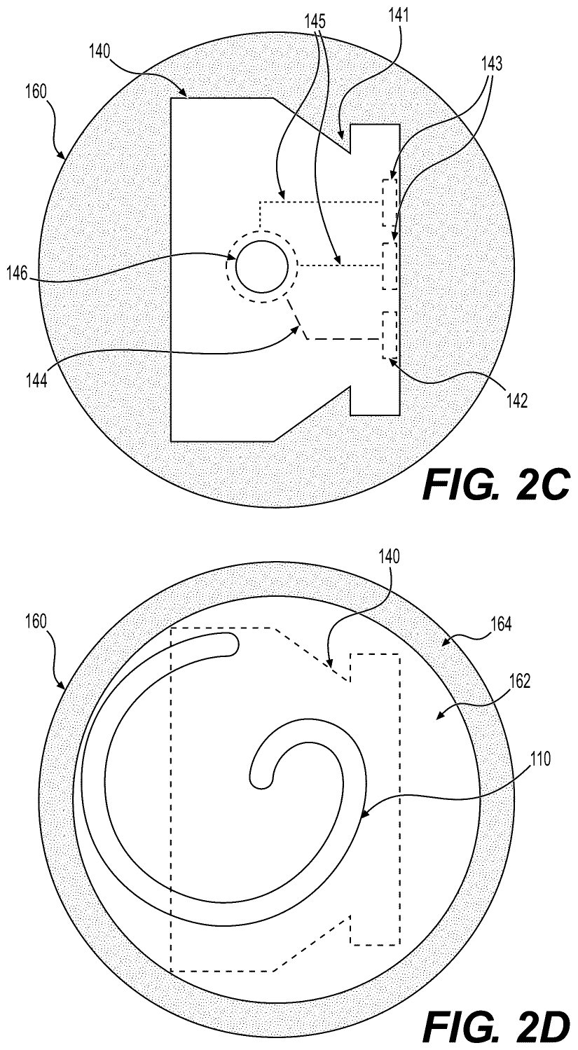

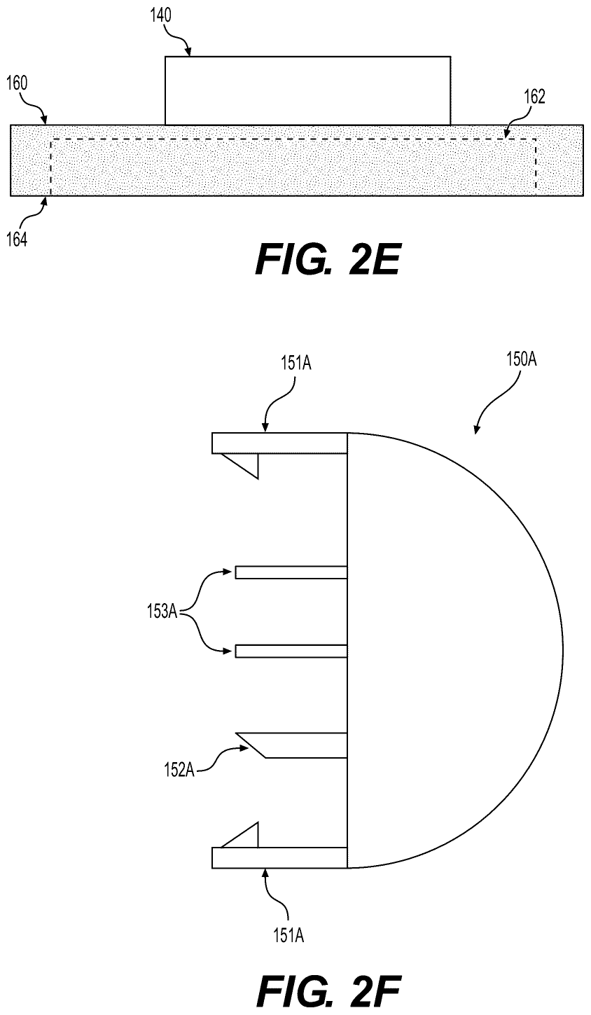

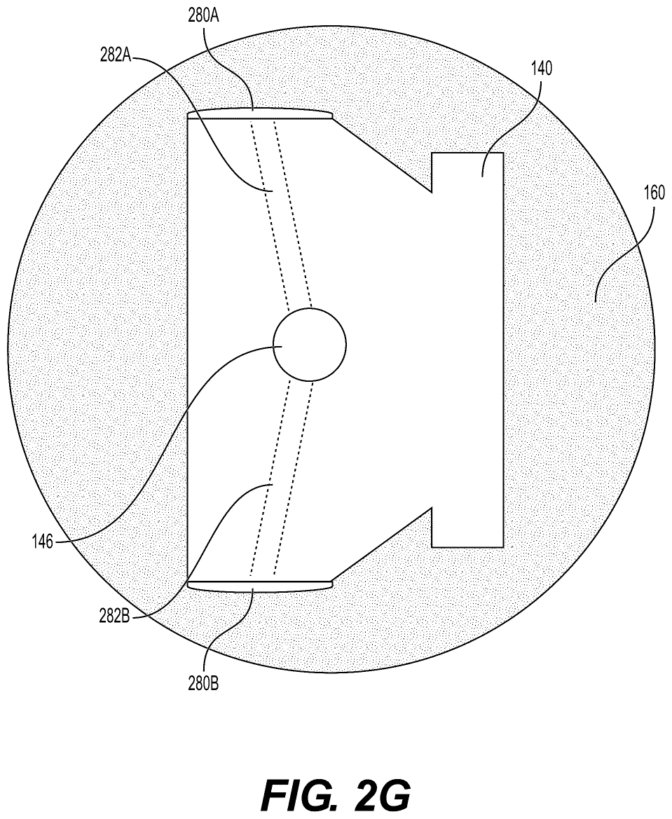

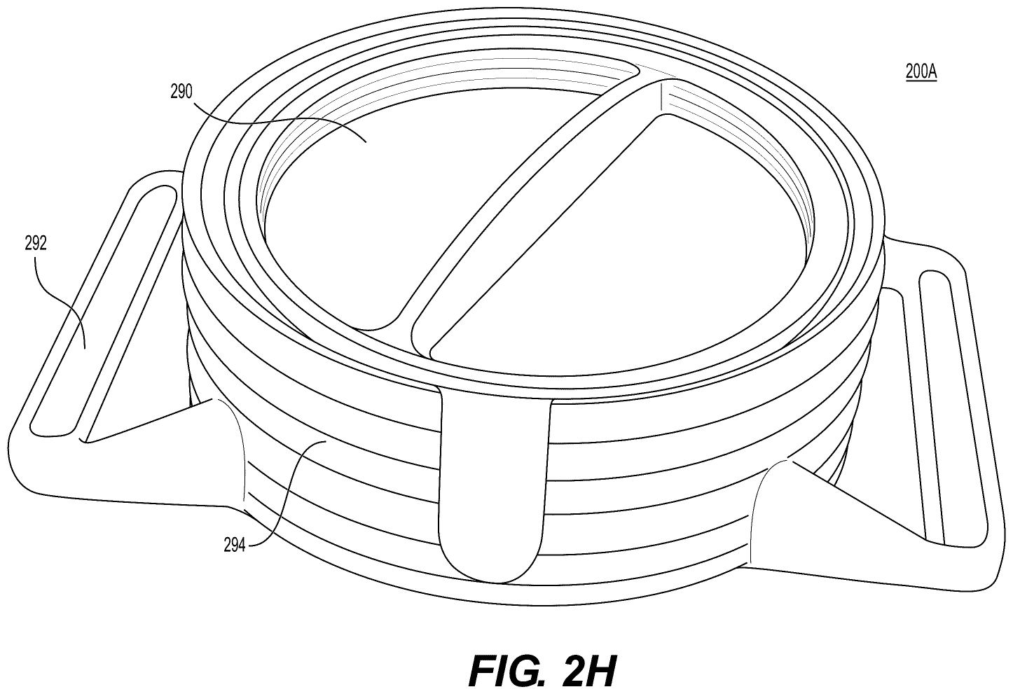

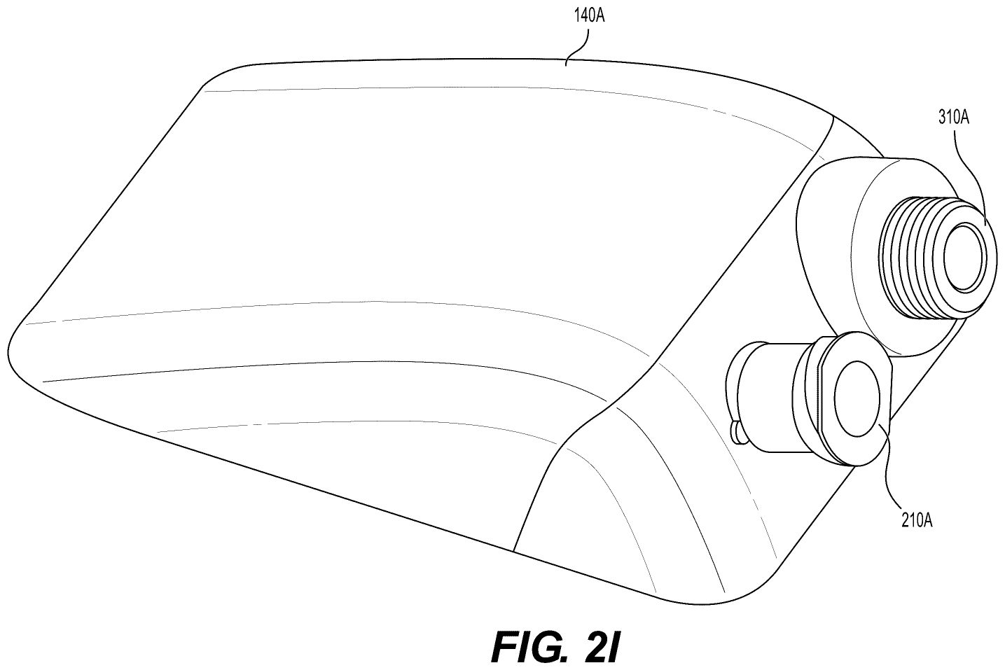

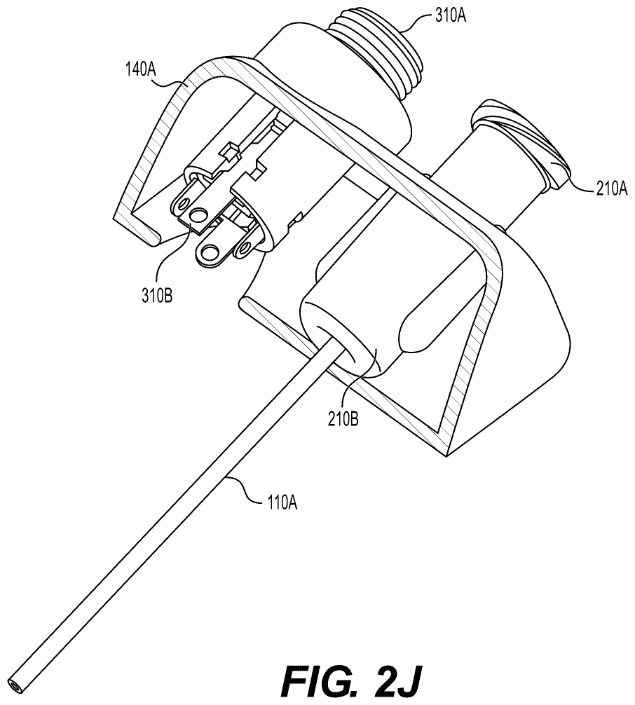

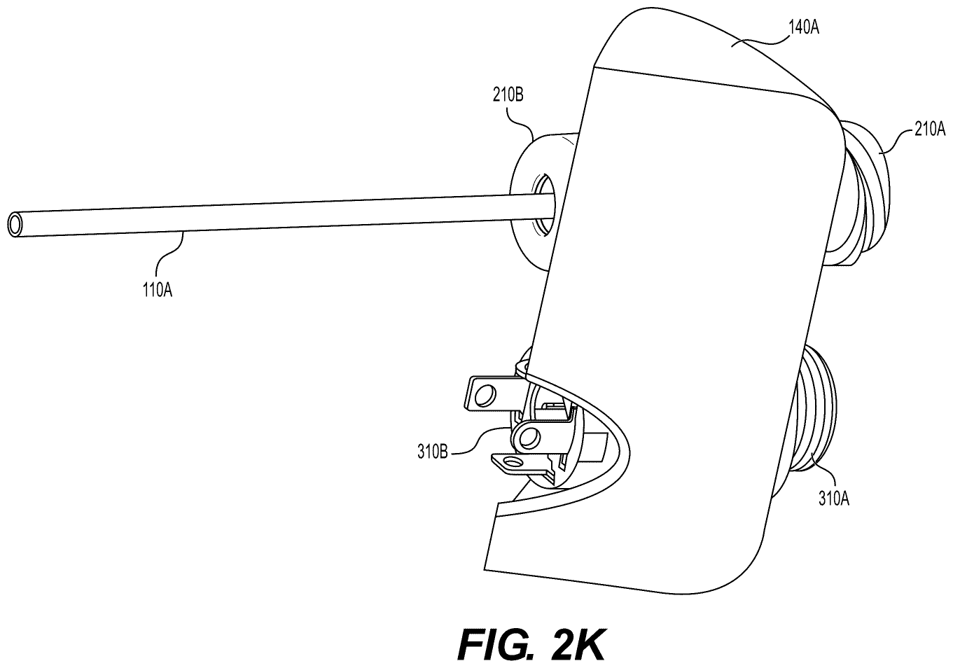

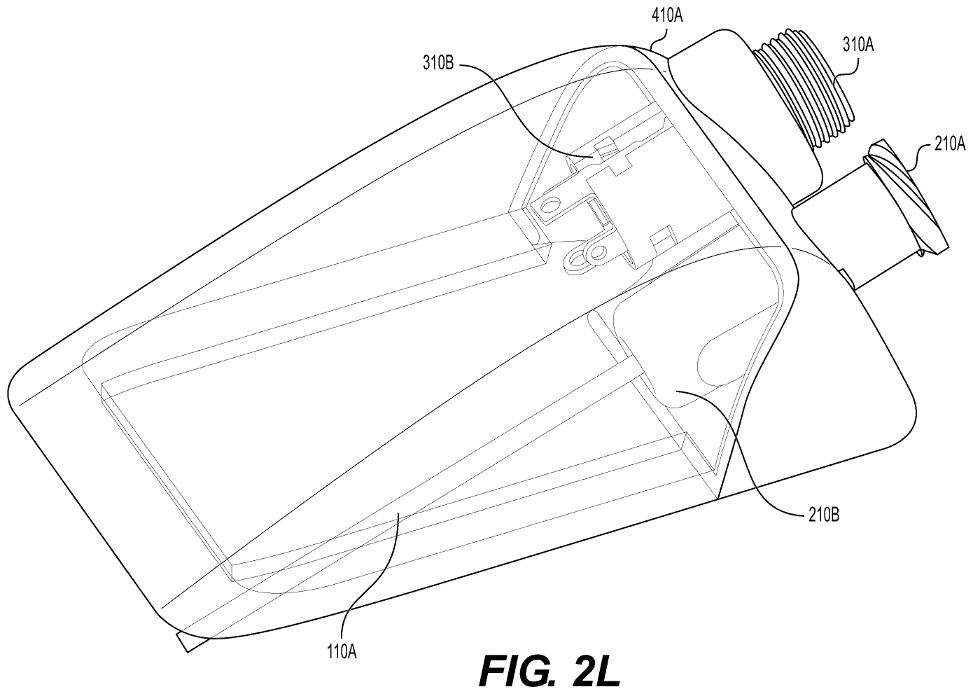

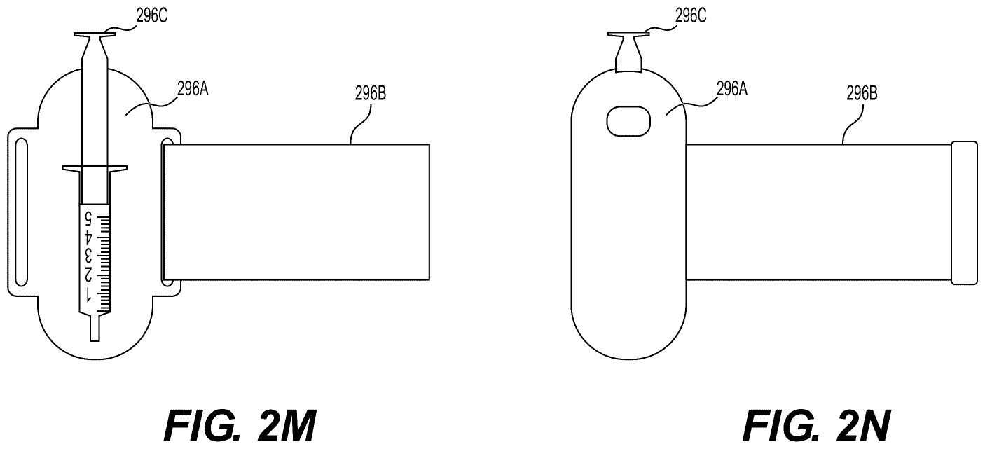

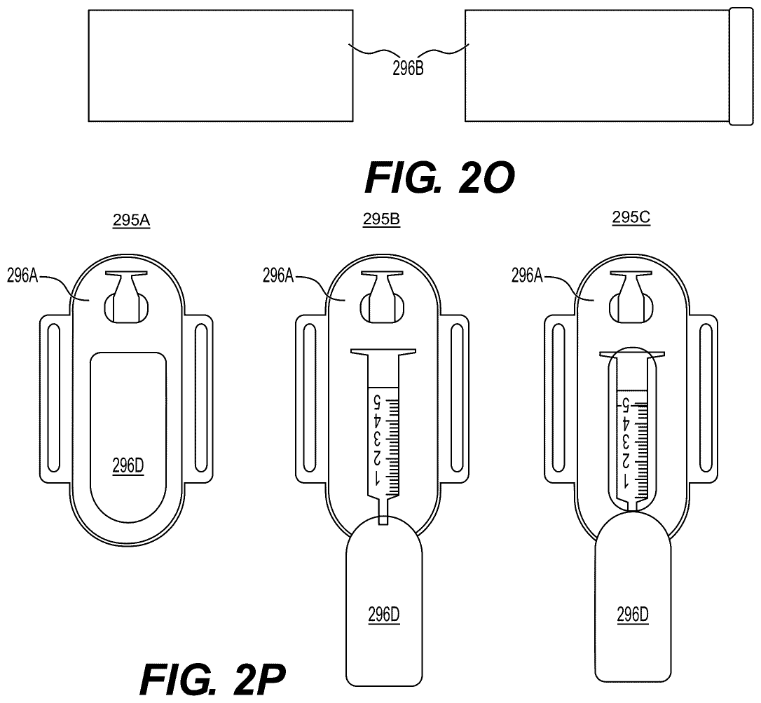

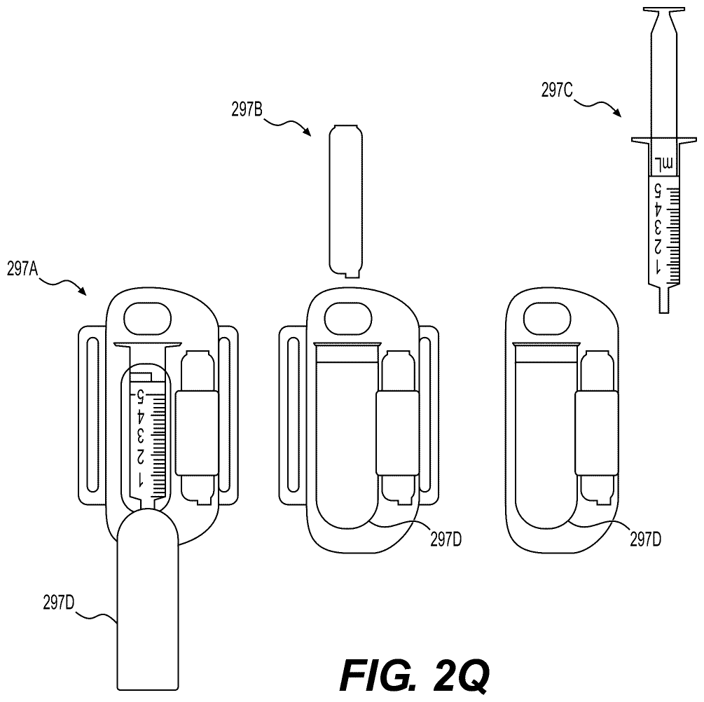

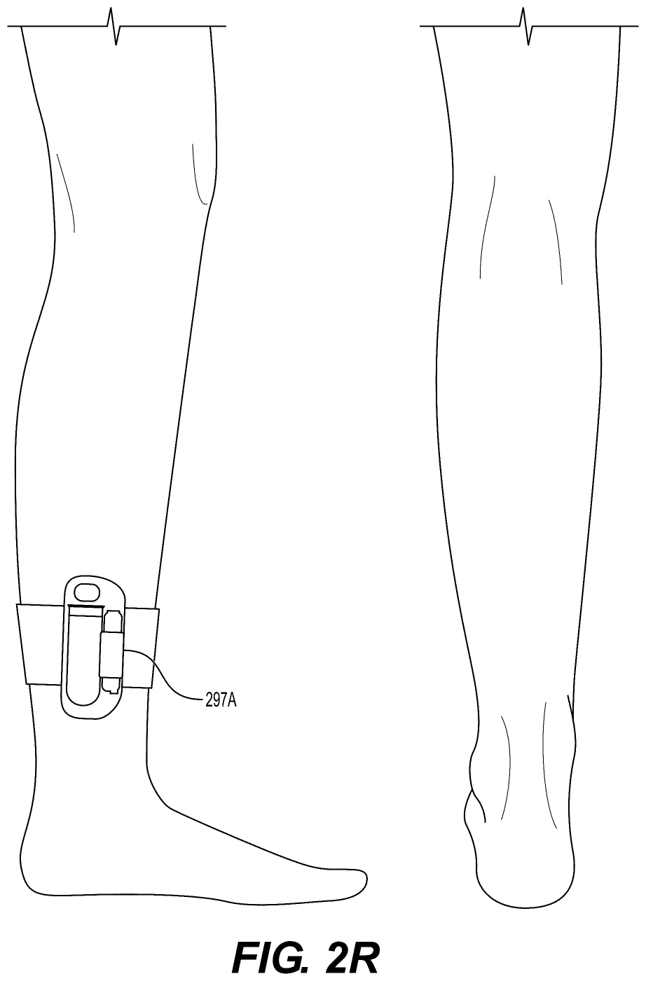

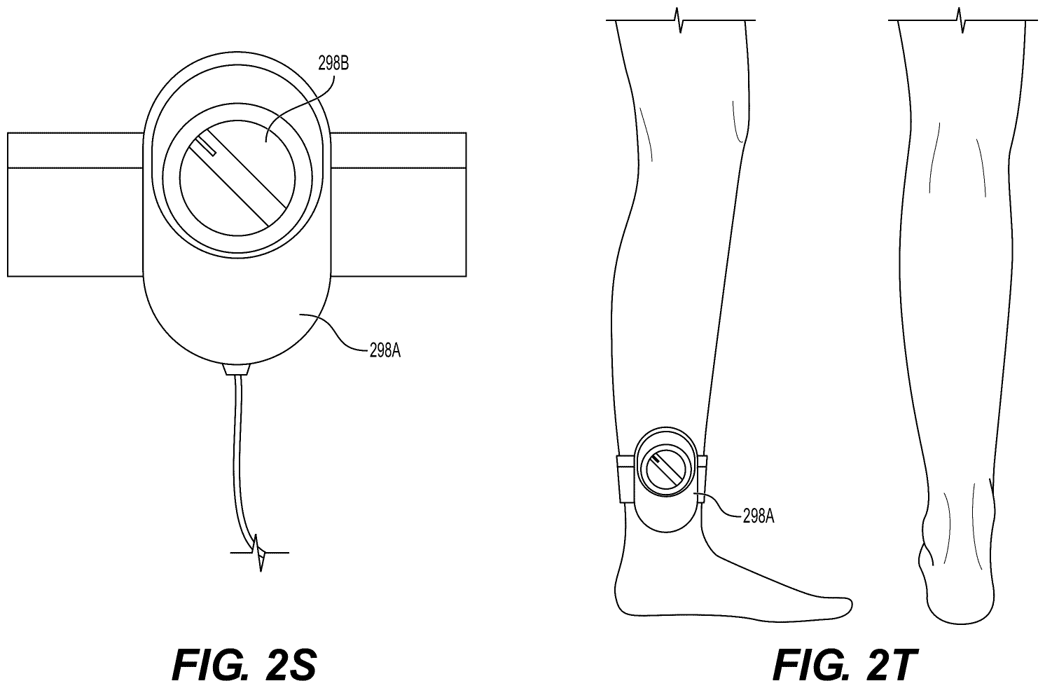

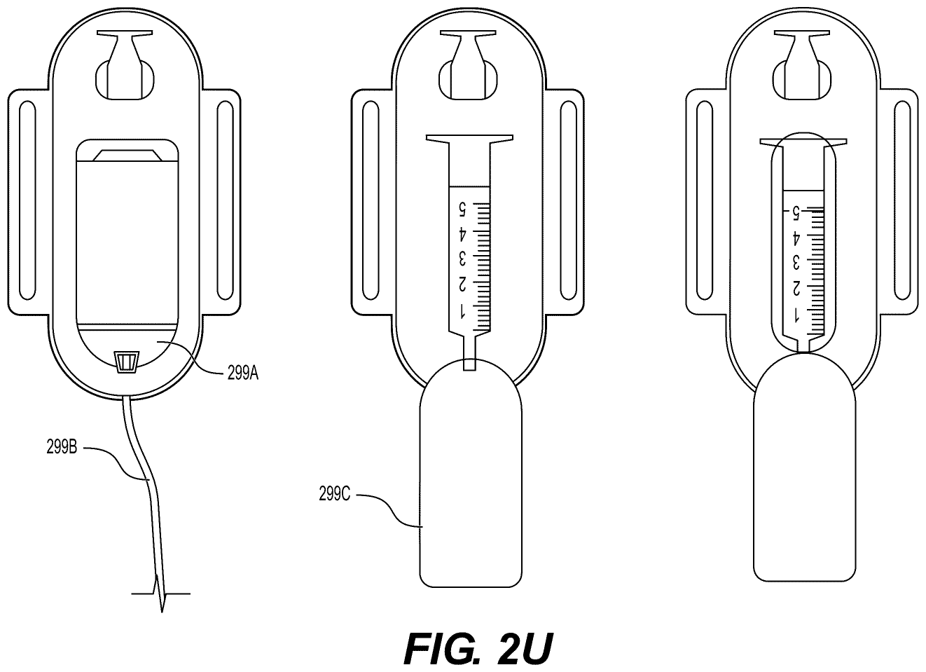

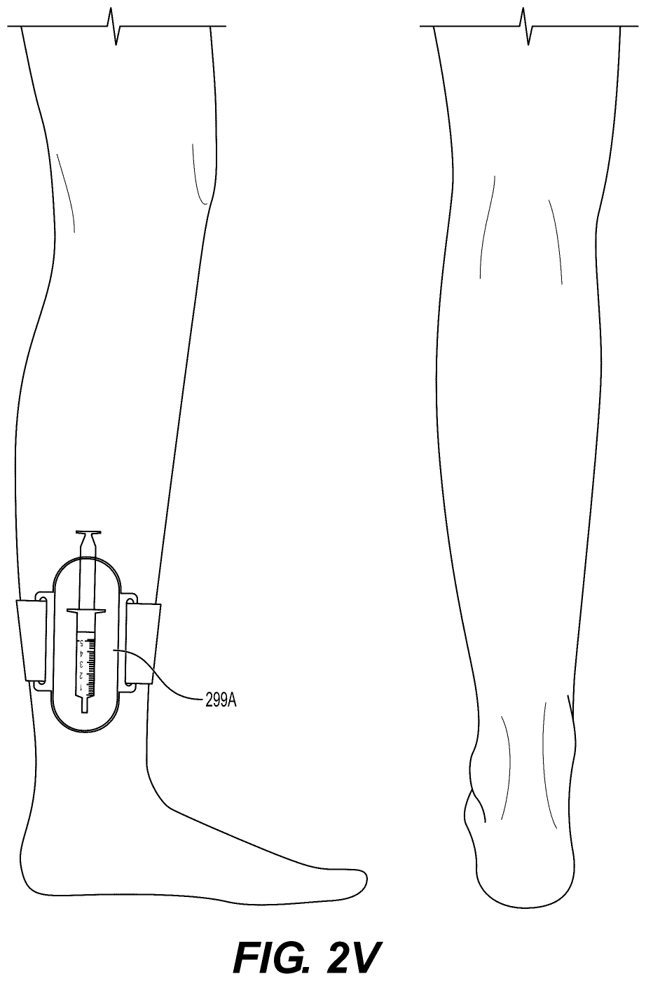

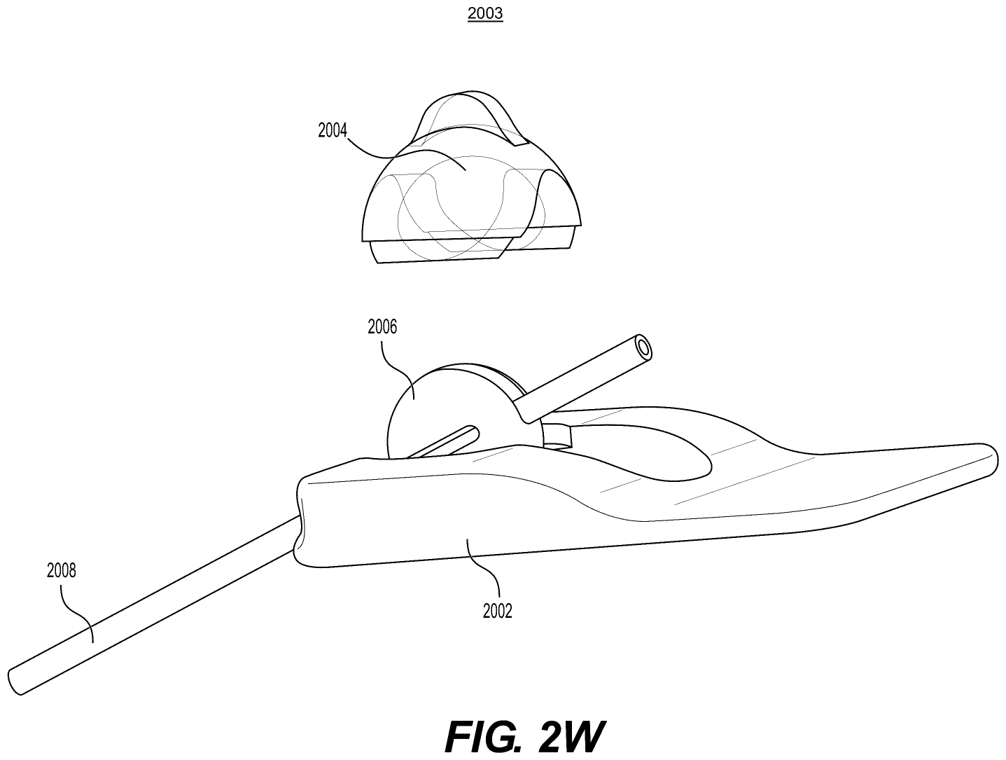

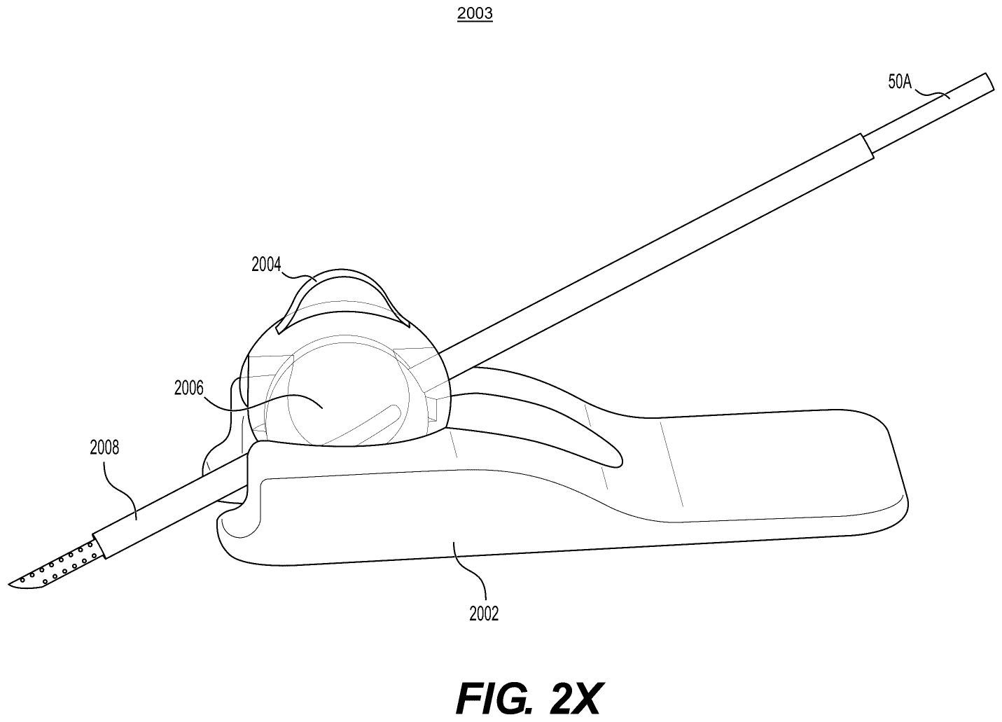

With reference to FIG. 2, a more detailed top-view schematic of housing 140 and connector 150 are shown. Connector 150 may have a pair of tabs 151 that provide snap-fit interlock with corresponding indents 141 in housing 140. It will be understood that connector 150 may connect or attach to housing 140 in any applicable manner including, but not limited to, the snap-fit interlock show in FIG. 2, via another snap-fit connection, via a force connection, via a fastener, or the like or a combination thereof. Connector 150 may detach from housing 140 via any applicable manner (e.g., a threshold amount of force, a release mechanism, a button or other input, etc.). Connector 150 may detach from housing 140 without dislocating and/or disturbing infusion lead body 110. Connector 150 may also include an insert molded hypodermic needle 152 in fluid communication, via an internal lumen 154, with infusion tube 210, as well as one or more metal pins 153 in electrical communication, via internal conductive traces 155, with cable 310. Similarly, housing 140 may include a hypodermic needle receptacle 142 and one or more pin receptacles 143, each with a corresponding gasket to form a fluid seal. Metal pins 153, internal conductive trances 155, pin receptacles 143, and/or conductive trances 145 may include electrical surfaces to transmit electrical signals across each respective component. The gaskets may be combined with one or more in-line filters to mitigate the ingress of bacteria. For example, an in-line filter may include or may be coated with anti-bacterial material or coating. A lumen 144 in the housing 140 provides fluid communication with the internal lumen of infusion lead body 110, and thus fluid communication with exit ports 118. Similarly, conductive traces 145 in housing 140 provide electrical communication with electrodes 112, 114. When connector 150 engages housing 140 (e.g., by sliding towards housing 140), the male fittings on the connector 150 functionally engage with the corresponding female fittings in the housing 140, thus functionally connecting the infusion lead body 110 to the drug pump 200 and/or pulse generator 300.

The housing 140 may further include a gasket 146 to provide a fluid seal around an insertion needle (described elsewhere herein) and subsequently close when the insertion needle is removed. The connector 150 may be configured for electrical stimulation only as shown in FIG. 2A, or the connector 150 may be configured for chemical stimulation only as shown in FIG. 2B. As shown in FIG. 2A, according to an embodiment, connector 150 may be manufactured or adjusted to include internal conductive traces 155, one or more metal pins 153, and a pair of tabs 151 and/or any other applicable connecting elements. As shown in FIG. 2B, according to an embodiment, connector 150 may be manufactured or adjusted to include insert molded hypodermic needle 152, internal lumen 154, and a pair of tabs 151 and/or any other applicable connecting elements. According to an embodiment, connector 150 may be a replaceable component such that the connector 150 of FIG. 2, connector 150 of FIG. 2A, connector 150 of FIG. 2B, and/or cap 150A of FIG. 2F may be interchangeable and/or disposable. Alternatively, or in addition, connector 150 may be modular such that one or more components of connector 150 (e.g., one or more metal pins 153, internal conductive traces 155, insert molded hypodermic needle 152, or internal lumen 154) may be attached to or detached from connector 150.

FIGS. 2C-2E schematically show housing 140 and a variant of adhesive patch 160 in more detail. FIG. 2C is a top view, FIG. 2D is a bottom view, and FIG. 2E is a side view. In this embodiment, adhesive patch 160 may include an open space 162 surrounded by perimeter 164 having a thickness that is greater than the infusion lead body 110. With this arrangement, excess length of the infusion lead body 110 may be arranged (e.g., in a spiral fashion), for example, inside the open space 162. The upper portion of the open space 162 may comprise an adhesive surface to secure the infusion lead body 110 (e.g., in its spiral configuration), and the bottom portion of the perimeter 164 may comprise an adhesive surface to secure the assembly to the skin. This configuration allows the infusion lead body 110 with its associated electrodes 112 and exit ports 118 to be placed in the desired lengthwise position (e.g., parallel to a nerve) independent of the length of the infusion lead body 110 while mitigating migration thereof. Additionally, this configuration allows the release of the excess length of infusion lead body 110 if housing 140 is accidentally detached from a user's skin. For example, if housing 140 is accidentally detached from a user's skin, release of the excess length of infusion lead body 110 may mitigate or prevent the portion of infusion lead body 110 inside the user's body from dislodging and/or or being pulled out.

According to an embodiment, excess length of the infusion lead body 110 may be cut or otherwise removed from a remaining portion of the infusion lead body 110 (e.g., the portion inserted into a patient's body). An excess proximal portion of the infusion lead body 110 may be cut using any applicable technique such as by using a blade or sharp surface, a laser, a perforation, or the like, or a combination thereof. The remaining portion of infusion lead body 110 may be connected to housing 140. For example, lumen 144 of housing 140 may be connected to the internal lumen of the remaining portion of infusion lead body 110. Similarly, conductive traces 145 of housing 140 may be connected to the internal wire of infusion lead body 110. This configuration allows the infusion lead body 110 with its associated electrodes 112 and exit ports 118 to be placed in the desired lengthwise position (e.g., parallel to a nerve) independent of the an original length of the infusion lead body 110, while mitigating migration thereof.

FIG. 2F schematically shows cap 150A. Cap 150A may connect to housing 140 when connector 150 is not connected to housing 140. Connector 150 may be detached when electrical stimulation or chemical stimulation is not required (e.g., during activity). Cap 150A may be connected to housing 140 to mitigate infection risk by preventing contaminants (e.g., bacteria) from entering hypodermic needle receptacle 142 and one or more pin receptacles 143.

Cap 150A may connect to housing 140 in any applicable manner that provides a full or partial seal between hypodermic needle receptacle 142 one or more pin receptacles 143 and the environment. Cap 150A may have a pair of tabs 151A that provide snap-fit interlock with corresponding indents 141 in housing 140. Cap 150A may also include a lumen insert 152A shaped to fit in lumen 144 and trace inserts 153A shaped to fit in conductive traces 145. Lumen insert 152A and/or trace inserts 153A may include an anti-microbial treatment (e.g., a coating, a finish, a material, etc.) which may further mitigate risk of infection. Accordingly, lumen insert 152A and trace inserts 153A may be shaped to fit lumen 144 and conductive traces 145, respectively, such that the anti-microbial treatment can be applied to lumen 144 and conductive traces 145. Connecting cap 150A to housing 140 may mitigate risk of exposure to contaminants and the anti-microbial treatment provide via cap 150A may treat the gaskets, filters, openings, inner space, and/or inner surfaces of lumen 144 and/or conductive traces 145 to remove contaminants therefrom. Cap 150A may be stored or positioned in an anti-microbial component when disconnected from housing 140, such that the cap 150A anti-microbial treatment is restored while cap 150A is disconnected from housing 140. Alternatively, or in addition, the anti-microbial treatment of cap 150A may be periodically replenished by application of the anti-microbial treatment onto cap 150A (e.g., onto lumen insert 152A and trace inserts 153A). As discussed herein, a safety mechanism configured to facilitate chemical and/or electrical stimulation based on a threshold property may be included in or may be associated with one or more of cap 150, cap 150A, housing 140, and/or another component discussed herein.

FIG. 2G is a schematic illustration showing a top view of housing 140 and adhesive patch 160. Housing 140 may include pressure button 280A and pressure button 280B. When activated (e.g., pressed), pressure buttons 280A and 280B may cause pressure rods 282A and 282B, respectively, to apply a pressure at or around gasket 146. The pressure at or around gasket 146 may be transferred to needle 50, as further discussed herein. The pressure may cause needle 50 to be held in place such that needle 50 is not able to advance or retract from its current position. The pressure may cause needle 50 and infusion lead body 110 to remain approximately stationary relative to each other. Buttons 280A and 280B and pressure rods 282A and 282B may be operated using a single hand to prevent or mitigate movement of needle 50 relative to infusion lead body 110. It will be understood that although activating pressure buttons 280A and 280B is describe to apply a pressure, buttons 280A and 280B and/or pressure rods 282A and 282B may be configured to apply the pressure when buttons 280A and 280B are not activated and release the pressure when 280A and 280B are activated. According to this embodiment, one or more additional components such a lever, a hinge, a release, or the like may be used to covert an activation pressure (e.g., as applied to activate buttons 280A and 280B) to release the pressure applied by pressure rods 282A and 282B.

FIG. 2H is a schematic illustration of a drug pump 200A. Drug pump 200A may be the same as or similar to drug pump 200 as disclosed herein. Drug pump 200A may include a knob 290, one or more handles 292, and/or a drug container 294 housed at least partially within drug pump 200A. Drug container 294 may be configured to house a given amount of one or more drugs (e.g., approximately 20cc per drug). Knob 290 may manually or automatically rotate such that each rotation may expel a predetermined amount of a drug stored at drug container 294 (e.g., approximately 5cc). Operation of knob 290 may be controlled by a safety mechanism, as discussed herein, where the safety mechanism is configured to prevent accidental and/or excess rotation of knob 290 (e.g., prevent an excess volume of drug, prevent frequent administration of a drug beyond a threshold amount, etc.). Although a rotatable knob 290 is shown, it will be understood that any applicable activation mechanism may be used to expel an amount of drug stored at drug container 294. For example, the activation mechanism may be a button, a slider, an electronic input receiver, a digital input receiver, and/or the like. Additionally, although a single drug container 294 is shown, it will be understood that multiple drug containers 294 storing the same or different drugs may be provided. In an embodiment where multiple drug containers are provided, an activation mechanism for each drug container of the multiple drug containers may be provided. Each such activation mechanism may be configured to expel all or a portion of the drug contained within a respective drug container based on a respective activation using a respective activation mechanism.

Knob 290 may be activated by rotating knob 290 by a given amount. The given amount may be predetermined amount. For example, one or more locks (not shown) may apply a counterforce to the rotation force of knob 290 such that rotation of knob 290 is temporarily terminated, after a partial rotation of knob 290, as a result of the counterforce. The one or more locks may prevent or mitigate accidental rotation of knob and, thereby prevent accidental administration of an excess amount of drug. A predetermined amount (e.g., approximately 5cc) of drug contained in the drug container 294 may be expelled as result of the rotation of knob 290. Rotation of knob 290 may be reinitiated for a subsequent partial rotation, after the temporary termination, such that an additional amount of drug is expelled based on the subsequent partial rotation. Rotation of knob 290 may cause expelling of the drug contained in drug container 294 as a result of a pressure applied by rotation of knob 290, by an opening created as a result of the of rotation of knob 290, and/or the like.

Drug container 294 may be formed of any applicable material configured to contain the drug. Drug container 294 may be sealed such that the drug contained in drug container 294 is not expelled without activation of an activation mechanism, such as rotation of knob 290. Drug container 294 may be formed of any applicable material configured to contain a drug such as, but not limited to, polyvinyl chloride (PVC), plastic, glass, and/or the like. Drug container 294 may be a compartment, a bag, and/or the like. Drug container 294 may be configured to house a replicable drug holder (e.g., a replaceable bag) which may be replaced by a user (e.g., when all or most of the drug in a current drug holder is expelled). Drug container 294 and/or the drug holder may store an amount of one or more drugs corresponding to a given number of activations (e.g., approximately 4-6 activations).

According to an embodiment, an activation mechanism (e.g., knob 290) may be activated automatically based on an electronic signal generated by a controller (e.g., by an external controller as further discussed herein). The electronic signal may trigger a motor or other component configured to cause activation of the activation mechanism. The electronic signal may further indicate a degree of activation of the activation mechanism. For example, the degree of activation may cause an amount of corresponding activation (e.g., rotation). The amount of corresponding activation may result in a corresponding amount of drug expelled from drug container 294 such that a higher amount of activation may expel a higher amount of drug.

One or more handles 292 may protrude from drug pump 200A and may be positioned against a user's body part such that the base of drug pump 200A and/or one or more handles 292 provide stability when positioning drug pump 200A against the user's body part. Alternatively, or in addition, a strap (not shown) may be extended through the one or more handles 292. The strap may extend through the one or more handles 292 as well as a user's body part (e.g., an ankle, a shoulder, an arm, a leg, etc.) such that drug pump 200A is secured to the user's body part via the strap. As an example, the strap may be self-securing (e.g., a Velcro™ strap) such that a portion of the strap attaches to another portion of the strap.

FIG. 2I is a schematic illustration of another housing 140A, according to an example embodiment of the present disclosure. Housing 140A may be the same as, similar to, or different than housing 140, as discussed herein. Housing 140A may include a rear portion including an infusion tube port 210A and a cable port 310A. According to an implementation, housing 140A may connect (e.g., directly) to pulse generator 300 via cable 310 and/or connect (e.g., directly) to drug pump 200 via infusion tube port 210A. For example, infusion tube port 210A may be configured to receive and/or connect to infusion tube 210. Cable port 310A may be configured to receive and/or connect to cable 310. According to another implementation, housing 140A may connect to a connector (e.g., connector 150, connector 150A, etc.). For example, infusion tube port 210A may connect to insert molded hypodermic needle 152 of connector 150 and cable port 310A may connect to internal lumen 154 of connector 150.

FIG. 2J is a schematic illustration of the interior view of housing 140A, FIG. 2K is a schematic illustration of a bottom portion of housing 140A, and FIG. 2L is a schematic illustration of a perspective view of housing 140A. As shown in FIGS. 2J-2L, housing 140A may include an interior infusion tube port 210B connected to and/or in fluid communication with infusion tube port 210A. Housing 140A may include an interior cable port 310B connected to and/or in electrical communication with cable port 310A. Internal infusion tube port 210B may include a lumen (e.g., lumen 144) in fluid communication with infusion tube 210, internal lumen 154, and/or an infusion lead body 110A. Infusion lead body 110A may be the same as or similar to infusion lead body 110, as discussed herein. A wire or electrical traces (not shown) may be provided to connect internal cable port 310B with an internal wire of infusion lead body 110A (e.g., via interior infusion tube port 210B) to provide electrical communication between cable 310 and wires (not visible) embedded in a wall of the infusion lead body 110A, as discussed herein in reference to infusion lead body 110. A bottom surface of housing 140A, shown in FIG. 2K, may be or include an adhesive (e.g., a foam backed adhesive) for securing housing 140A to a surface such as user's skin. An interior portion of housing 140A that includes the internal infusion tube port 210B and interior cable port 310B may be filled with an electrical isolation material (e.g., a potting material) to insulate the wires and/or electrical traces connecting internal cable port 310B with the internal wire of infusion lead body 110A. Infusion tube port 210A, internal infusion tube port 210B, cable port 310A, internal cable port 310B, and/or one or more components associated with housing 140A may include a safety mechanism as discussed herein, where the safety mechanism is configured to prevent accidental and/or excess output of chemical and/or electrical stimulation. As discussed, the safety mechanism may be facilitated using an electrical signal such that chemical and/or electrical stimulation is controlled in response to the electrical signal. For example, electrical activity via internal cable port 310B may be suspended for a given period of time, based on a signal generated by a safety mechanism. As another example, internal infusion tube port 210B may include a valve component that is configured to close for a given amount of time after a chemical stimulation is provided via internal tube port 210. A signal to initiate closer of the valve may be generated by the safety mechanisms and a signal to terminate closer of the valve may be generated by the safety mechanism or may be automatically generated, for example, based on a duration of time.

According to an implementation, one or more drugs may be stored within a strap which may be secured to a user's body part. The strap may be elastic and/or malleable (e.g., may have a softness or give above a threshold amount of softness or give), may be self-securing, and/or may be securable to a user's body part. The strap may be or may include a drug holder (e.g., an Intravenous (IV) bag approximately 2 inches by 6 inches in size) configured to expel one or more drugs. The drug holder may be connected to a one way check valve (e.g., forming a “T” shape) configured to expel a drug from the drug holder. For example, a syringe or prime bulb (e.g., having approximately 3cc of volume) may be in connection with the drug holder and configured to pull a drug from the drug holder through a tube in fluid communication with a user (e.g., through tubing 210) via the one way check valve. The drug holder as well as one or more of a tube, a valve, etc. may be embedded within or otherwise attached to the strap. Accordingly, the strap may configured to contain one or more drugs via a drug holder and the one or more drugs may be expelled to be received by a user from the strap. According to an implementation, the strap may include one or more drug holders which may hold a given amount of drug (e.g., approximately 100cc to approximately 500cc).

According to an implementation, one or more drugs may be stored within a wearable pump secured to a user's body (e.g., using a strap, using an adhesive, etc.). FIG. 2M is a schematic illustration of a wearable pump 296A and FIG. 2N is a back view of the wearable pump 296A, according to an example embodiment of the present disclosure. Wearable pump 296A may include drug holding component 296C shaped to store a drug (e.g., within a chamber), as shown in FIG. 2M. Drug holding component 296C may include a fluid container to hold a drug and may further include a retrieval component (e.g., a plunger) to expel and/or retrieve the drug from drug holding component 296C or into drug holding component 296C. Although FIG. 2M shows a retrieval component (e.g., plunger) that may be exposed past an upper surface of holding component 296C for ease of access, it will be understood that a retrieval component, some components, and/or all movable components of and/or associated with holding component 296C may be secured and/or inaccessible during an operational state and/or during a nonoperational state of a wearable pump such as wearable pump 296A. For example, as shown in FIGS. 2P, 2R, and 2U, as further discussed herein, a retrieval component and/or other movable component may not extend past the boundaries of the respective wearable pumps, mitigating unintentional contact with and/or accidental movement of such a component. Wearable pump 296A may be connected to one or more straps 296B, as shown in FIG. 2O. The one or more straps 296B may be configured to secure the wearable pump 296A to a user's body part, as discussed herein. Wearable pump 296A may be connected to a connector (e.g., connector 150, connector 150A, etc.) and/or may be directly connected to housing 140A to provide the drug to an infusion lead body (e.g., infusion lead body 110, infusion lead body 110A, etc.).

FIG. 2P is a schematic illustration of wearable pump 296A having cover 296D, according to an example embodiment of the present disclosure. FIG. 2P shows the cover 296D in a closed position at 295A, shows cover 296D in an open position from a back view at 295B, and shows the cover 296D in the open position from a front view at 295C. Cover 296D may cover all or a portion of holding component 296C. For example, cover 296D may cover all or portion of holding component 296C such that a user may not accidentally touch, move, or otherwise unintentionally interact with holding component 296C. According to an implementation, cover 296D may be shaped to include a chamber (not shown) to store a drug. According to this implementation, when cover 296D is in the open position, the holding component 296C may retrieve a given amount of drug from cover 296D (e.g., from a chamber of cover 296D). For example, in operation, a drug may be expelled from holding component 296C while cover 296D is in a closed position. Cover 296D may be transitioned to the open position and an additional amount of drug may be retrieved from cover 296D (e.g., from a chamber of cover 296D) into holding component 296C (e.g., by activating a plunger component associated with holding component 296C, causing a suction pressure to transfer an amount of drug from a chamber of cover 296D into holding component 296C).

FIG. 2Q is a schematic illustration of another wearable pump 297A, according to an example embodiment of the present disclosure. Wearable pump 297A may be similar to wearable pump 296A. Wearable pump 297A may be configured to receive one or more cartridges 297B containing one or more drugs therein. Cartridges 297B may be interchangeable such that a user may remove a first cartridge 297B from wearable pump 297A and may insert a second cartridge 297B into wearable pump 297A. As shown, one or more cartridges 297B may be stored within a storage component of wearable pump 297A. As also shown, cartridges 297B may be inserted into and/or removed from wearable pump 297A while a cover 297D is in an open or closed position. According to an implementation, instead of cartridges 297B, a holding component 297C may be inserted into wearable pump 297A. Alternatively, or in addition, holding component 297C may be used to refill one or more cartridges 297B. Wearable pump 297A may include a cover 297D, which may be similar to cover 296D discussed herein in reference to FIG. 2P.

FIG. 2R is a schematic illustration of a wearable pump 297A attached to a user, according to an example embodiment of the present disclosure. As shown, wearable pump 297A may be attached to a user body part (e.g., a leg). Wearable pump 297A may be attached to the user body part via an adhesive material (not shown). Alternatively, wearable pump 297A may be attached to the user body part via a strap (e.g., strap 296B).

FIG. 2S is a schematic illustration of another wearable pump 298A, according to an example embodiment of the present disclosure. Wearable pump 298A may be similar to drug pump 200A discussed in reference to FIG. 2H. Wearable pump 298A may include an activation mechanism such as a knob 298B which may be similar to knob 290 of FIG. 2H. FIG. 2T is a schematic illustration of wearable pump 298A attached to a user body part. Wearable pump 298A may be attached to the user body part via an adhesive material (not shown). Alternatively, wearable pump 298A may be attached to the user body part via a strap (e.g., strap 296B).

FIG. 2U is a schematic illustration of another wearable pump 299A, according to an example embodiment of the present disclosure. Wearable pump 299A may be similar to wearable pump 296A and/or wearable pump 297A. Wearable pump 299A may be connected to an infusion tube 298B which may be similar to infusion tube 210, as discussed herein. Wearable pump 299A may include a cover 299C, which may be similar to cover 296D discussed herein in reference to FIG. 2P. As shown in FIG. 2V, infusion tube 298B may be removable from wearable pump 299A such that while wearable pump 299A is not in operation, infusion tube 298B may be removed from wearable pump 299A. As also shown in FIG. 2V, wearable pump 299A may be attached to a user body part. Wearable pump 299A may be attached to the user body part via an adhesive material (not shown). Alternatively, wearable pump 299A may be attached to the user body part via a strap (e.g., strap 296B).

According to an implementation, infusion lead body 110 and/or infusion lead body 110A may be positioned to be substantially parallel to a nerve, thus spacing the two modes of stimuli along a length of the nerve where they do not interfere with each other. Methods for achieving this position are described with reference to FIGS. 3A-3F, as further discussed herein. According to another implementation, infusion lead body 110 may be positioned within a nerve sheath of a nerve that innervates a surgical site. Methods for achieving this position are described with reference to FIGS. 3H-3M, as further discussed herein.

FIG. 2W is a schematic illustration of a needle guide system 2003, according to an example embodiment of the present disclosure. Needle guide system 2003 may be used to position infusion lead body 2008 (e.g., similar to infusion lead body 110 and/or infusion lead body 110A) substantially parallel to a nerve, as described with reference to FIGS. 3A-3F and/or within a nerve sheath of a nerve as described with reference to FIGS. 3H-3M. Needle guide system 2003 may include a needle guide base 2002 that is connected to a needle guide 2006. Needle guide 2006 may be configured to receive an infusion lead body 2008 and needle 50A (e.g., similar to needle 50 discussed herein), as shown in FIG. 2X. Needle guide base 2002 may be positioned over a user's skin proximate to an insertion point where needle 50A is inserted into the user's body. A needle guide cover 2004, as shown in FIG. 2W and FIG. 2X, may be positioned on needle guide 2006. Needle guide 2006 may be rotatable around a first axis (e.g., substantially parallel to the needle guide base 2002), such that rotation of needle guide 2006 around the first axis may change the angle of insertion at which needle 50A is inserted at the insertion point. For example, rotation of needle guide 2006 around the first axis may cause the angle of insertion to change from approximately 85 degrees to approximately 5 degrees. The angle of insertion may be adjustable while needle guide cover 2004 is not positioned on needle guide 2006. The angle of insertion may be locked when needle guide cover 2004 is positioned on needle guide 2006. Once the angle of insertion is locked when needle guide cover 2004 is positioned on needle guide 2006, the angle may not change as needle 50A and/or infusion lead body 2008 is inserted into the insertion point (e.g., due to unintended movement). Accordingly, a practitioner may lock an angle of insertion and, once locked, may implement the techniques disclosed herein in reference to FIGS. 3A-3F and/or FIGS. 3H-3M using one hand, without risk of the angle of insertion changing. The angle of insertion may be determined based on the intended position of the infusion lead body 2008 such that a first intended position may require a first angle (e.g., 35 degrees) and as a second intended position may require a second, different, angle (e.g., 45 degrees). The intended position may be based on, for example, a nerve site, a body area for treatment (e.g., shoulder area, leg area, ankle area, etc.).

One or more drugs, as disclosed herein, may cause a regional anesthesia block for a vein proximate to the point of delivery (e.g., proximate to exit ports 118) of the one or more drugs, resulting in pain relief. Alternatively, or in addition, the one or more drugs may cause vasodilation of the tissue, vein, and/or nerve (e.g., resulting in a vein diameter over approximately 3 mm), which may result in AVF maturation. AVF maturation may correspond to the ability of an inflow artery and the vein to respond to increased blood flow that occurs upon anastomosis of the artery and vein. The duration of the AVF maturation may exceed the duration of the vasodilation, such that a given amount of drug may result in vasodilation for a first period of time, and may cause AVF maturation for a second period of time, where the second period of time is greater than the first period of time. The vasodilation and/or AVF maturation may provide clinical benefits such as for vein grafts, improved wound healing, reduced infection rate, and/or the like based at least on increased blood flow over a period of time. For example, expelling a drug over a period of approximately five days may catalyze healing by a given amount. Accordingly, vasodilation and/or AVF maturation, as discussed herein, may be used for pain management benefits, may facilitating ease of insertion or vein selection during a procedure (e.g., vasodilation may provide larger veins and/or better vein selection for an improved acute result), may increase blood flow, and/or the like. According to an implementation, chemical stimulation via the one or more drugs may be used to treat chronic wound healing and/or chronic pain relief (e.g., for complex regional pain syndrome (CRPS), dry gangrene, etc.).

According to an embodiment, an amount, frequency, or duration of stimulation may be determined to cause a given amount of vasodilation and/or AVF maturation (e.g., any vasodilation and/or AVF maturation) without providing pain relief. For example a first amount, frequency, and/or duration of chemical stimulation may result in vasodilation and/or AVF maturation whereas a second greater amount, frequency, and/or duration of chemical stimulation may result in pain relief in addition to vasodilation and/or AVF maturation.

As disclosed herein, one or more drugs may be provided via drug pump 200/200A. The one or more drugs may include, but are not limited to any applicable anesthetic, ropivacaine, bupivacaine, markain, lidocaine, dextrose, etc. A given drug may be selected based on the conductivity or lack of conductivity of the given drug. The amount of drug expelled via drug pump 200/200A may depend on the target nerve, tissue surrounding the target nerve, type of drug, and/or the target chemical stimulation (e.g., anesthetic effect, vasodilation, AVF maturation, etc.). For example, when targeting the tibial nerve, approximately 5cc may be expelled (e.g., as a single dose, per hour, per predetermined period of time, per activation, etc.). As another example, when targeting a shoulder nerve, 5cc may be expelled per hour. According to an implementation, a first initial amount of drug may be expelled at a first time (e.g., a first activation) and a second amount of drug may be expelled at a second time subsequent time. As further disclosed herein, the one or more drugs may be expelled as a bolus and/or may be expelled based on pulsed dosing (e.g., 3-5cc per period of time such as per approximately 3-4 hours). The amount of drug and/or frequency of activation may result in a corresponding result (e.g., approximately 6-8 hours of pain relief, approximately 8-10 hours of pain relief, approximately 10-12 hours of pain relief, a duration and/or amount of vasodilation, a duration and/or time of AVF maturation, etc. or a combination thereof). The duration of time and/or frequency that the one or more drugs are expelled may be based on a corresponding amount of battery life for a battery powering drug pump 200/200A and or external controller, as further discussed herein.

According to an embodiment, chemical or electrical stimulation may be triggered, modified, and/or updated based on user input or may be triggered automatically. User input may be received via a user device (e.g., a mobile device, computer, wearable device, etc.) and/or via an input component associated with any device or component disclosed herein. For example, a user may provide user input via a button or interface associated with housing 140, drug pump 200, pulse generator 300, and/or the like or a combination thereof. Such user input may be an instruction to trigger, modify, or update chemical or electrical stimulation, may be feedback regarding existing chemical or electrical stimulation, may be a user pain indication (e.g., a pain score), and/or the like.

Chemical and/or electrical stimulation may be conditioned on user input. For example, to increase compliance for user entered data, chemical and/or electrical stimulation may be administered in response to a user input. According to this example, the chemical and/or electrical stimulation may be triggered based on any user input, so long as there is a user input. For example, one or more properties (e.g., amount, frequency, duration, etc.) of chemical and/or electrical stimulation, as discussed herein, may or may not be determined based on the user input. However, the chemical and/or electrical stimulation may be withheld until a user input is received. According to an implementation, chemical and/or electrical stimulation may be conditioned on a specific user input based on a specific treatment. For example, receipt of a pain score may be required before administering (e.g., automatically) a bolus drug delivery. As another example, updated pain scores may be required for administering continued drug delivery (e.g., basal delivery).

Automatic stimulation may be triggered based on an event, an algorithmic output, or a machine learning output. An event based trigger may be based on, for example, a pain indication provided by a user, based on a change in a detected impedance, and/or the like. For example, one or more impedance sensors may be in contact with a vein, nerve, or tissue and may generate impedance signals. Upon determining an impedance greater than a threshold impedance, based on the impedance signals, an event based trigger may cause automatic stimulation (e.g., chemical and/or electric stimulation). An algorithmic output may be generated based on one or inputs (e.g., a user input indicating pain or a pain amount, detected impedance, etc.) such that an amount, frequency, and/or duration of chemical or electrical stimulation is triggered based on application of the inputs to an algorithm. Machine learning outputs are further discussed herein.

To avoid potential interference between the anesthetic solution and stimulation (either by dispersion of the electric field in the anesthetic solution or by blocking the Sodium-Potassium receptor on nerve cell or neuron), when the two modes of nerve block are administered simultaneously or in near-time, exit ports 118 and electrodes 112 may be spaced apart along the length of infusion lead body 110. However, to maintain the same or similar efficacy of the chemical and electrical stimuli, infusion lead body 110 may be positioned to be substantially parallel to the nerve as shown, thus spacing the two modes of stimuli along a length of the nerve where they do not interfere with each other. Methods for achieving this position are described with reference to FIGS. 3A-3F. However, other techniques may be employed such as alternating the delivery of chemical and electrical stimuli on a temporal basis such that electrical stimulation is delivered after the anesthetic solution has been substantially dispersed. For example, electrical stimuli could be delivered during the day, and chemical stimuli could be delivered during the night. This may be advantageous because chemical stimuli may interfere with motor function, whereas electric stimuli may not interfere with motor function. This would allow a patient to have physical therapy during the day without having motor function compromised.

FIGS. 3A-3F schematically illustrate example methods for positioning infusion lead body 110 substantially parallel to the nerve for the reasons stated above. The example methods of FIGS. 3A-3F may be implemented using the needle guide system 2003 of FIG. 2W and FIG. 2X. Aspects of this method may be similar to how local anesthetic is administered to achieve nerve block, namely the use of an appropriately sized needle 50 (e.g., a hypodermic needle) under ultrasonic guidance. It will be understood that although ultrasonic guidance is generally discussed herein, any applicable guidance technique (e.g., using radar, optical devices, sensors, etc.) may be used instead of and/or in addition to ultrasonic guidance. Needle 50 may include a proximal hub and a distal end (e.g., a sharped point) and may have a length suitable for extending through the gasket 146 in the housing 140, through infusion lead body 110, with the distal end of needle 50 extending out the distal end of infusion lead body 110. Needle 50 is also substantially more rigid than infusion lead body 110, thereby maintaining a straight configuration until removed. The diameter of needle 50 may be selected to closely match the inside diameter of the infusion lead body 110 to allow free movement therebetween but avoid a substantial gap that may otherwise compromise insertion through the skin.

Ultrasonic guidance may be used to locate a nerve (N) and/or a peripheral vein (V) or artery, adjacent to which a peripheral nerve (N) resides. The infusion lead assembly 100 may be pre-loaded onto the needle 50 such that the needle 50 extends through gasket 146 in housing 140 and out the distal end of the infusion lead body 110. According to an embodiment, buttons 280A and 280B may be activated or deactivated to pre-load infusion lead assembly 100 onto needle 50. Under continued ultrasound guidance, needle 50 and pre-loaded infusion lead assembly 100 may be inserted through the skin(S) as shown in FIG. 3A, until the distal tip of the needle 50 is adjacent the nerve (N). The hub of the needle 50 may be pushed toward the skin(S), without advancement of the needle 50, as shown in FIGS. 3B and 3C, to urge the assembly 100 into substantially parallel alignment with the nerve (N). This step involves displacing the nerve (N) and surrounding tissue in the opposite direction of the pushing force as opposed to cutting through tissue. With the infusion lead body 110 generally running parallel with the nerve, the needle 50 and infusion lead assembly 100 may be advanced under continued ultrasound guidance to avoid damage to the nerve (N) and avoid puncturing the vein (V) as shown in FIG. 3D. According to an implementation, infusion lead body 110 may be advanced past the needle tip of needle 50 without substantial movement of needle 50. Accordingly, needle 50 may surround a proximate portion of the infusion lead body 110 while a distal portion of the infusion lead body 110 extends past the tip of needle 50. Once the infusion lead assembly 100 is fully advanced with the adhesive patch 160 engaging the skin(S), the paper backing of the adhesive patch 160 may be removed, and then the needle 50 may be removed from the infusion lead assembly 100 as shown in FIG. 3E. According to an embodiment, buttons 280A and 280B may be activated or deactivated to remove needle 50 from infusion lead assembly 100. As the needle 50 is removed, the flexible nature of the infusion lead body 110 and the absence of the stiff needle 50 allows the nerve (N) and surrounding tissue to relax to its resting state, thereby resulting in a curved proximal portion of the infusion lead body 110, a relatively straight portion of the infusion lead body 110 running substantially parallel with the nerve (N), and the adhesive patch 160 securing the housing 140 to the skin directly over the insertion site to mitigate migration as shown in FIG. 3F.

According to an embodiment, infusion lead body 110 may be positioned within a nerve sheath of a nerve that innervates a surgical site. A nerve sheath is a layer of myelin and/or connective tissue that surrounds and insulates nerve fibers. FIG. 3G schematically shows infusion lead body 110 positioned to be substantially parallel to the nerve with a distal portion of infusion lead body 110 positioned within a nerve sheath (sheath). When inserted through and/or adhered to the epidermis (skin) and inserted in the sheath, as shown, the infusion lead body 110 may have a suitable length to position the distal electrodes 112 and exit ports 118 adjacent a nerve that innervates a surgical site. With this arrangement, a drug (e.g., anesthetic solution) may be delivered from the drug pump 200 to the nerve via exit ports 118, and electrical stimulation may be delivered from the pulse generator 300 to the nerve via electrodes 112, to provide combined chemical and electrical nerve block effects. A cover (e.g., having a valve, a slit, etc.) may be positioned over exit ports 118 and may mitigate or prevent occlusion of exit ports 118. The cover may be a silicone cover or may include bioresorbable or biodegradable material. The cover may be on an external surface of exit ports 118 or an internal surface of exit ports 118 (e.g., internal to infusion lead body 110).

Methods for achieving this position are described with reference to FIGS. 3H-3M. The example methods of FIGS. 3H-3M may be implemented using the needle guide system 2003 of FIG. 2W and FIG. 2X. Aspects of this method are similar to those described in FIGS. 3A-3F. Ultrasonic guidance may be used to locate a nerve, a nerve sheath, and/or a peripheral vein or artery, adjacent to which a peripheral nerve resides. The infusion lead assembly 100 may be pre-loaded onto the needle 50 such that the needle 50 extends through gasket 146 (not shown) in housing 140 and out the distal end of the infusion lead body 110. According to an embodiment, buttons 280A and 280B may be activated or deactivated to pre-load infusion lead assembly 100 onto needle 50. Under continued ultrasound guidance, needle 50 and pre-loaded infusion lead assembly 100 may be inserted through the skin as shown in FIG. 3H, until the distal tip of the needle 50 punctures the sheath, as shown. As shown in FIG. 3I, the distal end of infusion lead body 110 may also breach the sheath through the opening created by the tip of needle 50 puncturing the sheath. The hub of the needle 50 may be pushed toward the skin, without substantial advancement of the needle 50, as shown in FIGS. 3I and 3J, to urge the assembly 100 into substantially parallel alignment with the nerve while the distal tip of needle 50 and the distal end of infusion lead body 110 is between the sheath and the nerve. This step involves displacing the nerve and surrounding material (e.g., tissue, sheath material, etc.) in the opposite direction of the pushing force. As shown in FIGS. 3I-3L, the hub of needle 50 may be pulled away from infusion lead body 110 while infusion lead body 110 is pushed further into the sheath. Accordingly, the distal end of infusion lead body 110 may be inserted into the sheath without needle 50 being inserted into the sheath, mitigating the risk of needle 50 puncturing or damaging the vein or nerve. According to an embodiment, buttons 280A and 280B may be activated or deactivated to pull needle 50 away from infusion lead body 110. With the distal end of infusion lead body 110 positioned between the sheath and the nerve, and infusion lead body 110 generally running parallel with the nerve, infusion lead assembly 100 may be advanced under continued ultrasound guidance to avoid damage to the nerve and avoid puncturing the vein as shown in FIG. 3K. Once the infusion lead assembly 100 is fully advanced with the adhesive patch 160 engaging the skin, the paper backing of the adhesive patch 160 may be removed, and then the needle 50 may be fully removed from the infusion lead assembly 100 as shown in FIG. 3L. As the needle 50 is removed, the flexible nature of the infusion lead body 110 and the absence of the stiff needle 50 allows the nerve, nerve sheath, and surrounding tissue to relax to its resting state, thereby resulting in a curved proximal portion of the infusion lead body 110, a relatively straight portion of the infusion lead body 110 running substantially parallel with the nerve. As shown in FIG. 3M, electrodes 112 and exit ports 118 may be positioned within the sheath such that a drug (e.g., anesthetic solution) may be delivered from the drug pump 200 to the nerve via ports 118 within the sheath, and electrical stimulation may be delivered from the pulse generator 300 to the nerve via electrodes 112 within the sheath, to provide combined chemical and electrical nerve block effects. Adhesive patch 160 may secure the housing 140 to the skin directly over the insertion site to mitigate migration, as shown in FIG. 3M.

According to an embodiment, the sheath may be punctured or otherwise cut prior to inserting needle 50 and pre-loaded infusion lead assembly 100 through the skin and sheath, as shown in FIG. 3H. The sheath may be punctured or otherwise cut using any applicable technique such as a cutting device used to puncture or cut the sheath under ultrasound guidance. Accordingly, needle 50 and pre-loaded infusion lead assembly 100 may be inserted through the skin and the pre-punctured or pre-cut sheath, instead of the distal tip of needle puncturing the sheath in FIG. 3H.

According to an embodiment, electrodes 112 and exit ports 118 may be positioned proximate to each other such that both electrodes 112 and exit ports 118 are positioned inside the sheath, as shown in FIG. 3M. According to this embodiment, the amount of fluid (e.g., drug) delivered via exit ports 118 may be less than if exit ports 118 are positioned external to the sheath. Additionally, according to this embodiment, the size of exit ports 118 may be smaller than the size if exit ports 118 are positioned external to the sheath.

According to an embodiment, the distal end of infusion lead body 110 may be positioned such that electrodes 112 are positioned inside the sheath while exit ports 118 remain outside the sheath. According to this embodiment, infusion lead body 110 may still be positioned substantially parallel to the nerve, as disclosed herein. Electrical stimulation may be delivered from the pulse generator 300 to the nerve via electrodes 112 within the sheath, while a drug delivered from the drug pump 200 to the nerve is delivered outside the sheath.

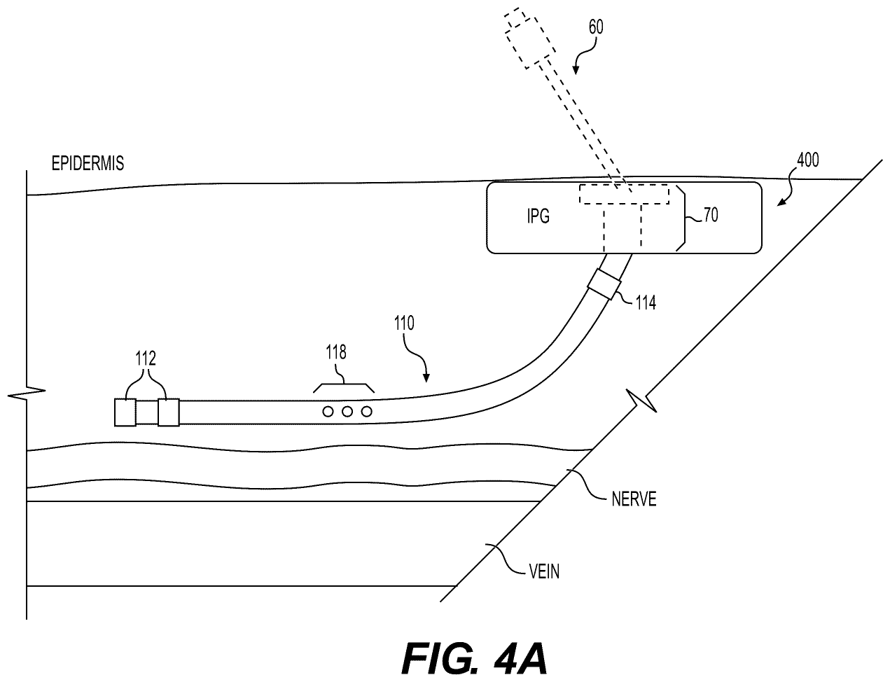

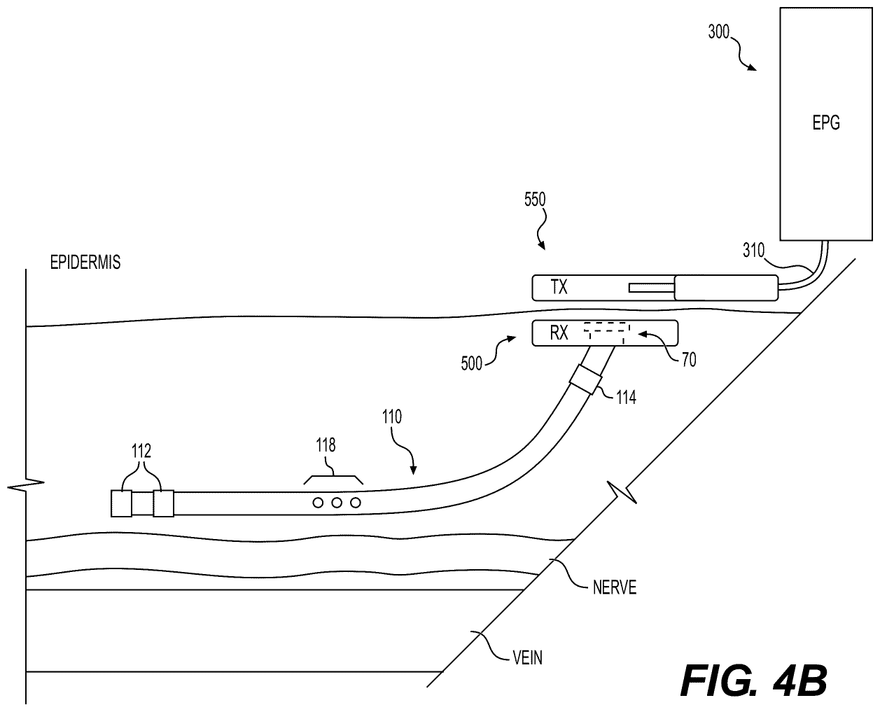

For sub-chronic (e.g., for less than approximately 60 days) and/or chronic pain management (e.g., for approximately 60 days or more), all or a portion of the infusion lead assembly 100, the drug pump 200 and the pulse generator 300 may be implanted under the skin. For example, an implantable pulse generator (IPG) 400 implanted under the skin may replace the housing 140 as shown in FIG. 4A. Alternatively, a combination of a receiver (RX) module 500 implanted under the skin and a wirelessly linked to a transmission (TX) module 550 (connected to an external pulse generator (EPG)) may replace the housing 140 as shown in FIG. 4B. A safety mechanism, as discussed herein, may be included in or associated with RX module 500 and/or TX module 550. The safety mechanism may be configured to prevent accidental and/or excess chemical and/or electrical stimulation in accordance with the techniques disclosed herein. For example, a safety mechanism may generate a signal to prevent electrical stimulation for a given period of time, in response to administration of electrical stimulation. After the given period of time, RX module 500, TX module 550, and/or the safety mechanism may generate a release signal allowing additional electrical stimulation. To facilitate the injection of an anesthetic drug via a needle 60 (e.g., hypodermic needle), a subcutaneous injection port 70 including a sealed gasket and lumen may be incorporated into IPG 400 or the RX module 500. In the embodiment of FIG. 4B, the RX module 500 may include a receiver antenna (e.g., coil) and/or any applicable inductive component to wirelessly receive a stimulation signal from the external pulse generator 300 wirelessly (e.g., RF or inductive) such that the stimulation signal is generated by the external pulse generator 300. Alternatively, the RX module 500 may include a stimulation circuit and a receiver antenna (e.g., coil) to receive power from the TX module 550 via a wireless link (e.g., RF or inductive) such that the stimulation signal is generated by the RX module 500.

In embodiments described herein, the components that are placed subcutaneously (excluding IPG 400 due to the battery contained therein) may comprise a bioresorbable or biodegradable polymer (e.g., infusion lead body 110) and/or a bioresorbable or biodegradable metal (e.g., electrodes 112, RX module 500, etc.). Such polymers and metals are described by Choi et al. in the article entitled “Fully implantable and bioresorbable cardiac pacemakers without leads or batteries”, Nature Biotechnology (2021), the entire disclosure of which is incorporated herein by reference. Other examples of biodegradable polymers include Polyglycolide or poly(glycolic acid) (PGA), poly(L-lactic acid) (PLLA), poly(3-hydroxybutyrate) (PHB), Polycaprolactone (PCL), or the like, or a combination thereof. Electrodes 112 and/or electrodes 114 may be may be wirelessly powered and may be controlled from outside a patient's body, as disclosed herein. Electrodes 112 and/or electrodes 114 may deliver electrical signals at up to approximately 30V-60V. An electrical signal delivered via electrodes 112 and/or electrodes 114 may be a pulse train with a given pulse width, frequency (e.g., approximately 50 kHz-40 kHz), and amplitude (e.g., approximately 0-15Vpp). According to an embodiment, a patient may adjust the amplitude and/or frequency via patient input provided via external pulse generator 300 and/or an external controller. For example, a patient may input an amplitude and/or frequency or may select an amplitude and/or frequency from two or more pre-programmed amplitudes and/or frequencies.

Table 1 shows example electrical parameters that may be output or applied by one or more electrical components disclosed herein such as electrical pulse generator 300, IPG 400, RX module 500, TX module 550, electrodes 112, and/or electrodes 114.

TABLE 1

| Parameter | Output |
|---|---|
| Pulse Frequency | Approximately 5-1500 Hz |
| Pulse Width | Approximately 50-500 microseconds |
| Current/Voltage Regulated | Current |
| Output Voltage (300 Ω) | Approximately 0-4.1 V |
| Output Voltage (500 Ω) | Approximately 0-6.4 V |
| Output Voltage (800 Ω) | Approximately 0-7.5 V |
| Output Current (300 Ω) | Approximately 0-13.5 mA |
| Output Current (500 Ω) | Approximately 0-12.8 mA |
| Output Current (800 Ω) | Approximately 0-9.4 mA |
| Waveform | Charge Balanced (delayed) Biphasic |
| | asymmetrical |
| Polarity | Programmable (Anode, Cathode, or |
| | Off) |
| Pulse Shape | Decaying Exponential |
| Avg. Current Density (300 Ω) | Approximately 105.0 mA/cm² |
| Avg. Current Density (500 Ω) | Approximately 95.1 mA/cm² |
| Avg. Current Density (800 Ω) | Approximately 69.0 mA/cm² |
| Max. Phase Charge* (300 Ω) | Approximately 6.8 μC/pulse |
| Max. Phase Charge* (500 Ω) | Approximately 6.4 μC/pulse |
| Max. Phase Charge* (800 Ω) | Approximately 4.7 μC/pulse |
| Max. Charge Density* (300 Ω) | Approximately 53.1 μC/ cm² |
| Max. Charge Density* (500 Ω) | Approximately 50.3 μC/ cm² |
| Max. Charge Density* (800 Ω) | Approximately 36.9 μC/ cm² |
| Max. Current Density* (300 Ω) | Approximately 106.1 mA/ cm² |
| Max. Current Density* (500 Ω) | Approximately 100.6 mA/ cm² |
| Max. Current Density* (800 Ω) | Approximately 73.9 mA/ cm² |
| Net Charge | Approximately 0 μC |
| Avg. Phase Power (300 Ω) | Approximately 0.053 W/phase |
| Avg. Phase Power (500 Ω) | Approximately 0.073 W/phase |
| Avg. Phase Power (800 Ω) | Approximately 0.062 W/phase |
| Avg. Phase Power Density (300 | Approximately 0.42 W/cm²/phase |
| Ω) | |
| Avg. Phase Power Density (500 | Approximately 0.58 W/cm²/phase |
| Ω) | |
| Avg. Phase Power Density (800 | Approximately 0.48 W/cm²/phase |
| Ω) | |
| Pulse Delivery Mode | Continuous |
| ON/OFF Times | No Cycling |
| Current Path Option | Bipolar |

Bioresorbable and/or Biodegradable materials may conduct energy until a threshold amount of degradation occurs, after which point the energy conduction may be limited or reduced below a given threshold (e.g., a usability threshold). Biodegradable material may degrade in tiers. For example, a first tier may be a first number of days (e.g., 5-12 days), where energy conduction (e.g., via an electrode) is reduced to a first conduction tier. The biodegradable material may degrade to one or more second tiers (e.g., 12-20 days, 12-40 days, 40-60 days, etc.), when the energy conduction is reduced to one or more second condition tiers and/or to no conduction. Non-biodegradable material may be used to insert and/or place the biodegradable material.

Degradation of biodegradable material may be accelerated or decelerated based on one or more of material and/or material amount selected for the biodegradable material, application of a degradation catalyst (e.g., amount or type of catalyst), an area of placement of biodegradable material within body, and/or the like. A receiver antenna (e.g., an antenna of RX module 500) may receive a wireless transmission at a given frequency range. If wireless transmission is received at the given frequency range, then energy may be generated at the electrode (e.g., based on the receiver antenna resonating).

The distance between the transmission coil and the receiving antenna may be determined based on length of time the biodegradable material is within a body (e.g., a longer length of time may require a shorter distance).

A control device (e.g., a mobile device, an external device, etc.) may be used to control an external device (e.g., TX module 550) that may be positioned on or near a user's skin (e.g., via a patch), via wired or wireless connection. The control device may cause a transmission coil to output a wireless pacing signal. The control device may determine properties of the wireless pacing signal based on one or more of: the location of the electrode, user attribute (e.g., level of pain, level of medication consumed (e.g., opioid), type of medication consumed, type of stimulation (e.g., neuro-stimulation), duration of time from past stimulation and/or drug consumption, pattern of past stimulation and/or drug consumption, or the like).

For example, a biodegradable electrode may be inserted into a patient's body and may provide electrical current to a portion of the patient's nervous system (e.g., spinal cord). The patient may undergo a procedure and may input a pain level. The control device may determine a pacing signal to be output by a wireless pacing device such that the electrode resonates to stimulate an area of the spinal cord to reduce the pain sensed by the user. Such stimulation may be used as a substitute for or to complement medication (e.g. pain medication).

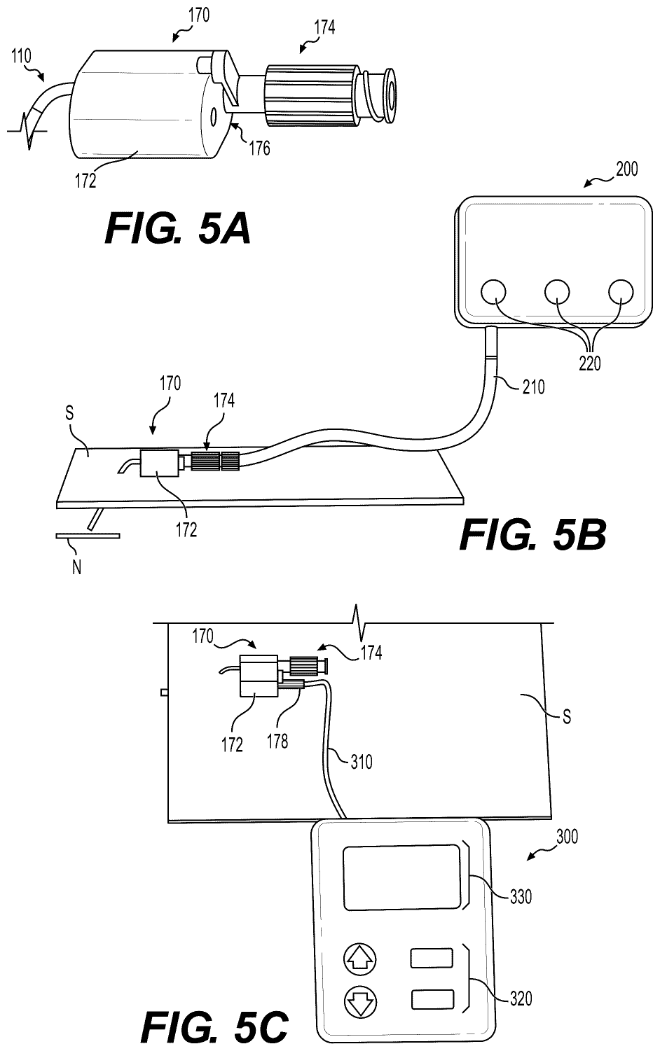

With reference to FIGS. 5A-5C, an alternative multimodal pain management system is shown schematically. In this embodiment, the alternative system includes a module 170 connected to an infusion lead body 110 (as described previously). Module 170 may include a housing 172, a fluid line connector 174 (e.g., touhy borst) and a female electrical receptacle 176, optionally sealed with a removable silicone rubber plug, for example. The fluid line connector 174 may include an in-line filter to mitigate the ingress of bacteria and/or pathogens, and may include a sealed diaphragm. The fluid line connector 174 may be removably attached to a drug pump 200 via tubing 210 to provide fluid communication from the drug pump 200 to the nerve (N) via infusion lead body 110 as described previously. Similarly, and the female electrical receptacle 176 may be removably connected to a male jack 178 and cable 310 for electrical connection to an external pulse generator 300 to provide electrical communication from pulse generator 300 to the nerve via infusion lead body 110 and associated electrodes as described previously. The module 170 and infusion lead body 110 may be covered by an adhesive patch attached to the skin(S) to secure the system in place. The alternative multimodal pain management system of FIGS. 5A-5C may be inserted adjacent to a nerve in accordance with the techniques described in reference to FIGS. 3A-3M.

The drug pump 200 may be an electromechanical pump (e.g., motor-controlled piston in chamber) or a mechanical pump (e.g., spring or manually operated syringe type or bulb), for example. As shown in FIG. 5B, the drug pump 200 may have a simple user interface that allows only a prescribed amount of drug (e.g., anesthetic) to be administered by the patient, thus allowing the drug to be delivered in controlled-volume discrete boluses, such as three boluses of approximately 5cc-10cc each, one or more boluses for transmission of 50cc-100cc per day, etc., for example. Each bolus may be activated by the user pressing a button 220, for example, to administer the drug as needed (e.g., in response to perceived pain). The other buttons may be locked-out for a period of time to prevent the delivery of multiple boluses simultaneously. The external pulse generator 300 may also have a simplified user interface that allows the patient to select from a limited number of prescribed pulse regimens (e.g., prescribed frequency and amplitude) using up/down, select and start buttons 320 and display screen 330.

Drug pump 200 may allow a periodic basal delivery of a prescribed amount of drug (e.g., anesthetic) to be administered throughout a period of time (e.g., a day, a night, on a continuous basis, etc.). The basal delivery may be activated by a user via a drug pump 200 interface or an external controller, as further discussed herein. The basal delivery may be implemented based on one or more pre-programmed basal delivery settings that may be input by a user or may be stored at drug pump 200 or an external controller. The basal delivery settings may be adjustable by a user or via a signal received at drug pump 200 or an external controller. Drug pump 200 may allow a bolus dose delivery based on patient activation. A patient may provide patient input to activate a bolus dose delivery via drug pump 200 and/or via an external controller. For example, drug pump 200 may be programmed to provide basal doses at a constant or variable levels. Additionally, the patient may provide patient input to trigger a bolus dose delivery, which may be greater in amount than the basal doses.

According to an embodiment, drug pump 200 and/or external pulse generator 300 may communicate with an external controller (e.g., a mobile device, a stand-alone device, etc.). Such communication may be wired or wireless. According to this embodiment, drug pump 200 may not include buttons 220 or may include a subset of the buttons 220. Similarly, external pulse generator 300 may not include buttons 320 or may include a subset of buttons 320. The external controller may include an interface (e.g., a graphical interface, a physical interface, etc.) that provides selectable components (e.g., buttons, icons, etc.). Selection of such selectable components may cause one or more signals to be transmitted by the external controller. The signals may be received at a receiver in communication drug pump 200 and/or external pulse generator 300. The one or more signals may cause drug pump 200 and/or external pulse generator 300 to perform the actions disclosed herein (e.g., activating drug pump 200 and/or pulse generator 300, causing drug pump 200 to output a given amount of fluid, causing pulse generator 300 to output an electrical signal, etc.). For example, the external controller may have a simple user interface that allows only a prescribed amount of drug (e.g., anesthetic) to be administered by the patient based on the user selecting a corresponding selectable component.

The external controller may include code, a script, and/or the like which may cause an external controller processor to generate the one or more signals. The one or more signals may be generated based on user input selecting one or more selectable components (e.g., via buttons, icons, etc.) and/or based on programmed instructions. For example, the external controller may be a mobile device having component (e.g., a transmitter) to wirelessly transmit the one or more signals (e.g., via Bluetooth, infra-red, WiFi, a local area network, a wide area network, etc.). An application or interface may be accessed using the mobile device (e.g., a web application, a mobile application, etc.) and the application may receive user inputs (e.g., an input to activate drug pump 200 and/or external pulse generator 300, an input to modify a drug dosage or frequency, an input to trigger electrical activity, etc.) via one or more selectable components of the application. The application may cause the mobile device to transmit the one or more signals based on a selected selectable component. The transmitted signal may be received by drug pump 200 and/or external pulse generator 300 and may cause drug pump 200 and/or external pulse generator 300 to perform an action.

The external controller may be programmable such that the one or more signals are generated based on pre-programmed settings. Such settings may automatically cause the external controller to transmit the one or more signals based on a trigger. The trigger may be a time, a duration of time, a sensor input, an external signal, or the like or a combination thereof.

According to an implementation, machine learning outputs of a machine learning model may control chemical and/or electrical stimulation. The machine learning model may be trained based on historical or simulated inputs and corresponding historical and/or simulated chemical and/or electrical stimulation. The inputs may include, but are not limited to, treatment properties (e.g., type of procedure, type or severity of injury or condition, etc.), user properties (e.g., user demographics, user weight, user biological properties, user pain thresholds, etc.), pain relief, vasodilation (e.g., amount vasodilation), AVF maturation (e.g., amount of AVF maturation), impedance, etc. The machine learning model may be trained by modifying one or more weights, layers, synapsis, nodes, or the like of the machine model, based on a machine learning algorithm, as further disclosed herein.

The trained machine learning model may receive current inputs associated with a user and may generate one or more outputs based on the same. For example, the machine learning model may receive treatment properties, user properties, pain relief properties, current or expected vasodilation information, current or expected AVF maturation information, and/or impedance values. The current inputs may be provided based on user input and/or one or more sensors configured to detect a respective current input. Based on the current inputs, the machine learning model may output a chemical or electrical stimulation properties such as chemical or electrical stimulation amount, frequency, and/or duration. The output may be based on a target time (e.g., target termination of electrical and/or chemical stimulation), a trend (e.g., reduction of electrical and/or chemical stimulation over time), and/or target electrical and/or chemical stimulation. The algorithmic and/or machine learning outputs discussed herein may facilitate a closed-loop system such that electrical and/or chemical stimulation and related properties (e.g., frequency, amount, duration, etc.) are automatically determined based on algorithmic and/or machine learning outputs. For example, electrical and/or chemical stimulation may be output based on an algorithmic and/or machine learning output schema which may be adjusted based on user input (e.g., pain scores). Electrical and/or chemical stimulation may be adjusted in accordance with the algorithmic and/or machine learning output schema based on trend analysis to, for example, wean the electrical and/or chemical stimulation over time (e.g., based on a user's response to such stimulation). The weaning may include reducing electrical and/or chemical stimulating without the user experiencing an adverse effect such as an increase in pain. The chemical or electrical stimulation properties may be provided to the external controller and external controller may be configured to trigger pump 200/200A and/or pulse generator 300 based on the chemical or electrical stimulation properties. The machine learning model may be configured to provide updated chemical or electrical stimulation properties based on updated current inputs.

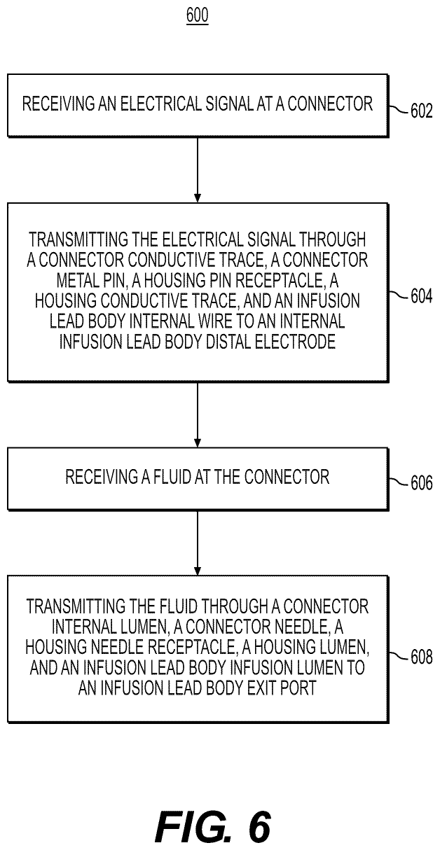

FIG. 6 is a flowchart 600 for multimodal electrical and chemical stimulation in accordance with the embodiments disclosed herein. At step 602, an electrical signal may be received at a connector (e.g., connector 150). The electrical signal may be received form a pulse generator (e.g., pulse generator 300) and may be generated at the pulse generator based on a user input, a pre-programmed setting, etc. At step 604, the electrical signal or one or more signals generated based on the electrical signal may be transmitted through an electrical path via connector conductive trace, a connector metal pin, a housing pin receptacle (e.g., of housing 140) a housing conductive trace (e.g., of housing 140), and/or an infusion lead body internal wire (e.g., of infusion lead body 110) to an internal lead body distal electrode (e.g., distal electrode 112).

At step 606, a fluid may be received at the connector. The fluid may be received from a pump (e.g., pump 200) and may be a drug or other chemical or fluid. The fluid may be received based on a user input, a pre-programmed setting, etc. At step 608, the fluid may flow through a fluid path via a connector internal lumen, a connector needle, a housing needle receptacle (e.g., of housing 140), a housing lumen (e.g., of housing 140), and/or an infusion lead body infusion lumen (e.g., of infusion lead body 110) to an infusion lead body exit port (e.g., exit ports 118).

The techniques described in flowchart 600 may be used to provide both electrical stimulation and chemical stimulation to a user. The electrical signal transmitted through the distal electrode at step 604 may provide electrical stimulation based on one or more electrical signal properties (e.g., frequency, amplitude, change in frequency or amplitude, phase, duration, etc.). The fluid transmitted through the exit port at step 608 may provide chemical stimulation based on chemical properties of the transmitted fluid.

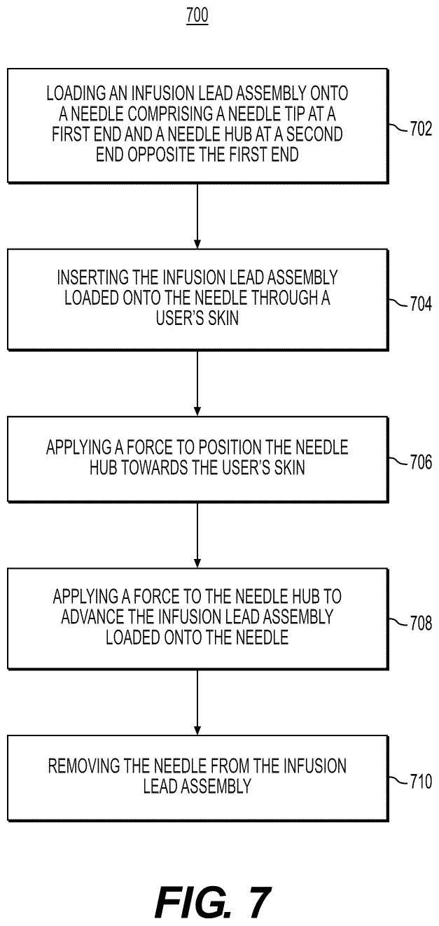

FIG. 7 is a flowchart 700 for infusion lead body placement in accordance with embodiments disclosed herein. At step 702, an infusion lead assembly (e.g., infusion lead assembly 100) may be loaded onto a needle (e.g., needle 50). The needle may include a needle tip at a first end and a needle hub at a second end opposite the first end. The infusion lead assembly may be loaded onto the needle by traversing the needle tip through a gasket (e.g., gasket 146) or other opening at an infusion lead assembly housing (e.g., housing 140). The needle tip may advance through an infusion lead body (e.g., infusion lead body 110) of the infusion lead assembly through a distal end of the infusion lead body.

At 704, as shown in FIG. 3A, the infusion lead assembly loaded onto the needle may be inserted through a user's skin. The needle may puncture the user's skin and the infusion lead assembly loaded onto the needle may advance through the opening created by the needle puncturing the user's skin. At step 706, as shown in FIGS. 3A-3C, a force may be applied (e.g., at the needle hub and/or the proximal portion of the infusion lead assembly) to position the needle hub towards the user's skin. The force at step 706 may be applied until the infusion lead assembly loaded onto the needle is substantially parallel to a nerve, as shown in FIG. 3C.

At step 708, as shown in FIG. 3D, a force may be applied to the needle hub to advance the infusion lead assembly loaded onto the needle further inside the user's body. The force applied at step 708 may be applied such that the infusion lead assembly loaded onto the needle is advanced further inside the user's body while the infusion lead assembly loaded onto the needle remains substantially parallel to the vein. At step 710, as shown in FIG. 3E, the needle may be removed (e.g., unloaded) from the infusion lead assembly. The needle may be removed by pulling or otherwise extracting the needle away from the infusion lead assembly such that the needle trip traverses the infusion lead assembly and exists via the proximal end of the infusion lead assembly. As shown in FIG. 3F, an infusion lead body (e.g., infusion lead body 110) of the infusion lead assembly may remain inside the user's body after the needle is removed from the infusion lead assembly. The infusion lead body may remain substantially parallel to the user's vein. The steps described in FIGS. 3A-FIG. 3F may be performed using a single hand. For example, at step 702, the infusion lead assembly loaded onto the needle may be inserted through a user's skin using a single hand. As another example, the force applied at step 708 may be applied by a single hand. Accordingly, he infusion assembly and/or needle may include gripping material and/or contours such that the steps described in FIGS. 3A-FIG. 3F may be performed using a single hand.

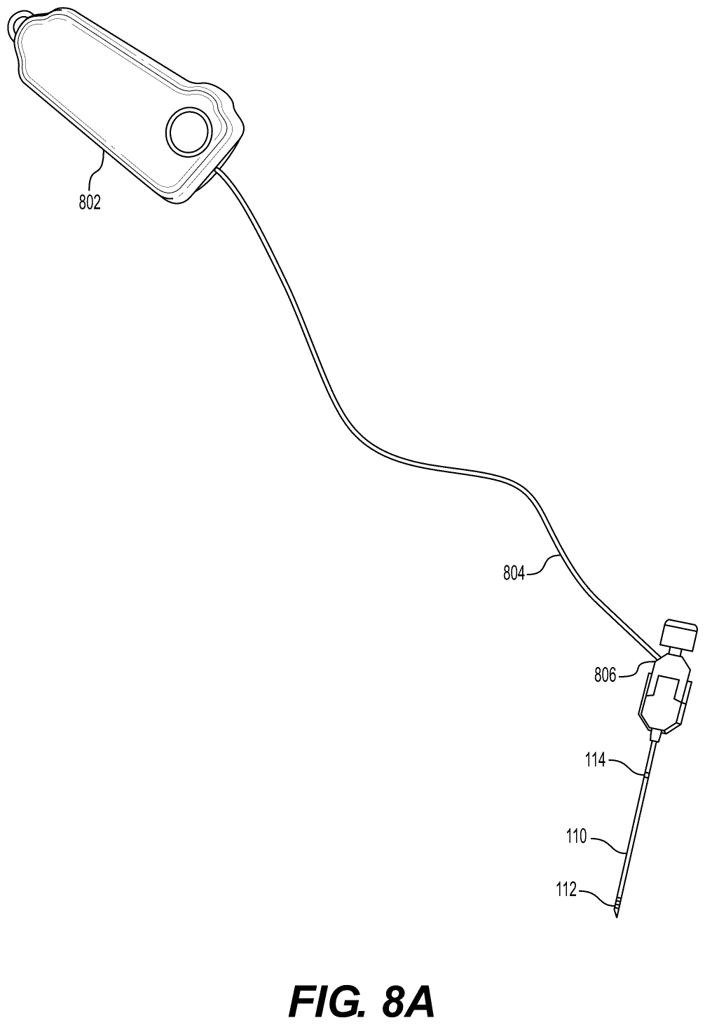

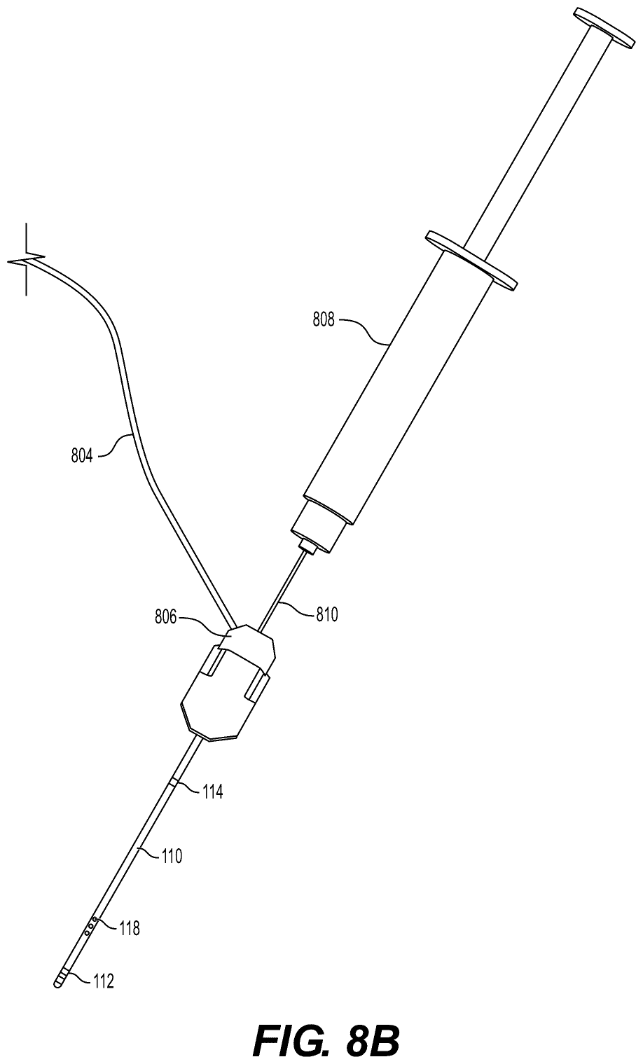

FIG. 8A is a schematic illustration of an electronic placement detector 802. Electronic placement detector 802 may be attached to placement connector 806 via a wire 804. Placement connector 806 may include one or more conductive traces. The conductive traces in placement connector 806 may connect with conductive traces 145 in housing 140 to provide electrical communication between electronic placement detector 802 and electrodes 112, 114. During insertion of infusion lead body 110 (e.g., as described in FIGS. 3A-3M) and/or after placement of infusion lead body 110 inside a patient's body, electronic placement detector 802 may be connected to housing 140 via wire 804 and placement connector 806.

Electronic placement detector 802 may generate low voltage electronic signals that are transmitted to electrodes 112 and/or electrodes 114 via an electrical path created by the electronic placement detector 802, wire 804, placement connector 806, conductive traces 145 in housing 140, and infusion lead body 110 internal wire. A patient and/or medical provider may trigger electronic placement detector 802 to generate low voltage electronic signals such that they are output via electrodes 112 and/or electrodes 114. Placement of infusion lead body 110 proximate to the nerve may be confirmed based on the patient reporting a sensation in response to the low voltage electronic signals. Alternatively, or in addition, the low voltage electronic signals may trigger a motor response and placement of infusion lead body 110 proximate to the nerve may be confirmed based on the observed motor response.

FIG. 8B is a schematic illustration of a visual placement detector 808. Visual placement detector 808 may be attached to placement connector 806 via a fluid channel 810. As shown in FIG. 8B, visual placement detector 808 may be connected to infusion lead body 110 at the same time as electronic placement detector 802 is connected to infusion lead body 110. Alternatively, either visual placement detector 808 or electronic placement detector 802 may be connected to insertion of infusion lead body 110 at a given time. Placement connector 806 may include a fluid channel. The fluid channel in placement connector 806 may connect with lumen 144 in housing 140 to provide fluid communication between visual placement detector 808 and exit ports 118. During insertion of infusion lead body 110 (e.g., as described in FIGS. 3A-3M) and/or after placement of infusion lead body 110 inside a patient's body, visual placement detector 808 may be connected to housing 140 via fluid channel 810 and placement connector 806.

Visual placement detector 808 may include a chamber or may be connected to a chamber that includes a detection medium. The detection medium may be any applicable fluid (e.g., saline solution) or gas (e.g., air) that may be inserted into a patient's body via exit ports 118. Visual placement detector 808 may provide the detection medium to exit ports 118 via a fluid path created by the visual placement detector 808, fluid channel 810, placement connector 806, lumen 144 in housing 140, and infusion lead body 110 infusion lumen. A patient and/or medical provider may trigger visual placement detector 808 to provide the detection medium via exit ports 118. Placement of infusion lead body 110 proximate to the nerve may be confirmed based on visual confirmation (e.g., via ultrasound) detection medium location as it exits exit ports 118 and comparing the detection medium location to the location of a given nerve.

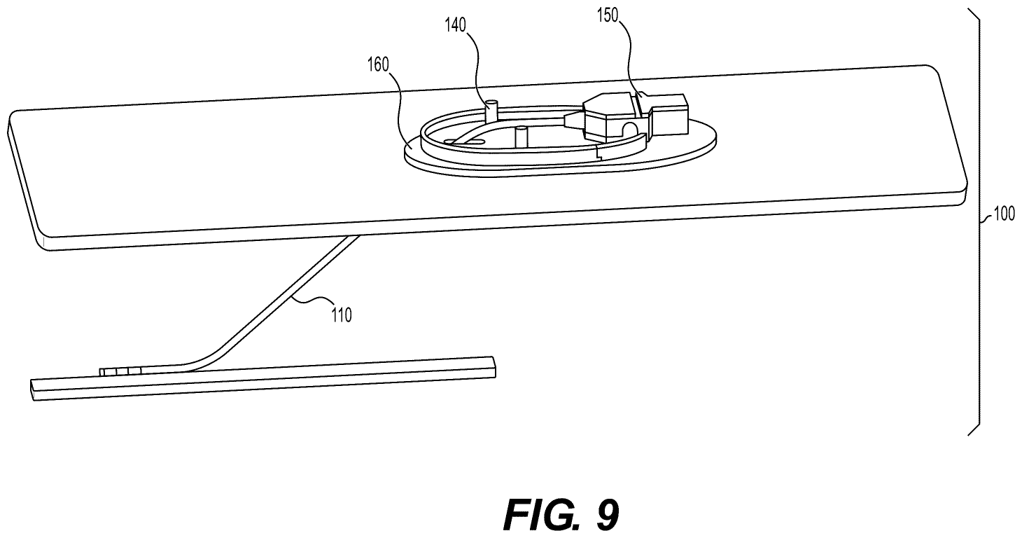

FIG. 9 is another schematic illustration of a multimodal system. The system may generally include an infusion lead assembly 100 configured for releasable connection to a drug pump 200 (shown in FIG. 1A), a pulse generator 300 (shown in FIG. 1B), or a combined drug pump and pulse generator 200/300 (shown in FIG. 1C) via a housing 140, a connector 150 and associated infusion tube and cable. The housing 140 may be secured to the epidermis via an adhesive patch 160 to mitigate migration. The top portion of the adhesive patch 160 may be attached (e.g., permanently) to the underside of the housing 140, and the bottom portion of the patch 160 may include an adhesive layer (e.g., suitable for approximately 10-14-day use under normal living conditions) covered by a removable covering (e.g., removable wax paper) until ready for application to the epidermis.

The infusion lead assembly 100 may include a tubular infusion lead body 110 having a proximal end connected to the housing 140. The infusion lead body 110 may include an infusion lumen (not visible) extending therethrough providing fluid communication between exit ports 118 (as shown in FIG. 1A) and the drug pump 200 via housing 140, connector 150, and infusion tube (e.g., when connector 150 is connected to housing 140). The infusion lead body 110 may further include one or more distal electrodes 112 (as shown in FIG. 1A) and one or more proximal electrodes 114 (as shown in FIG. 1A) in electrical communication with the pulse generator 300 via wires (not visible) embedded in the wall of the infusion lead body 110, via internal wires (not shown) extending through the housing 140 and connector 150, and via the cable. The internal wires embedded in the wall of the infusion lead body 110 may extend alongside at least a portion of the infusion lumen of infusion lead body 110.

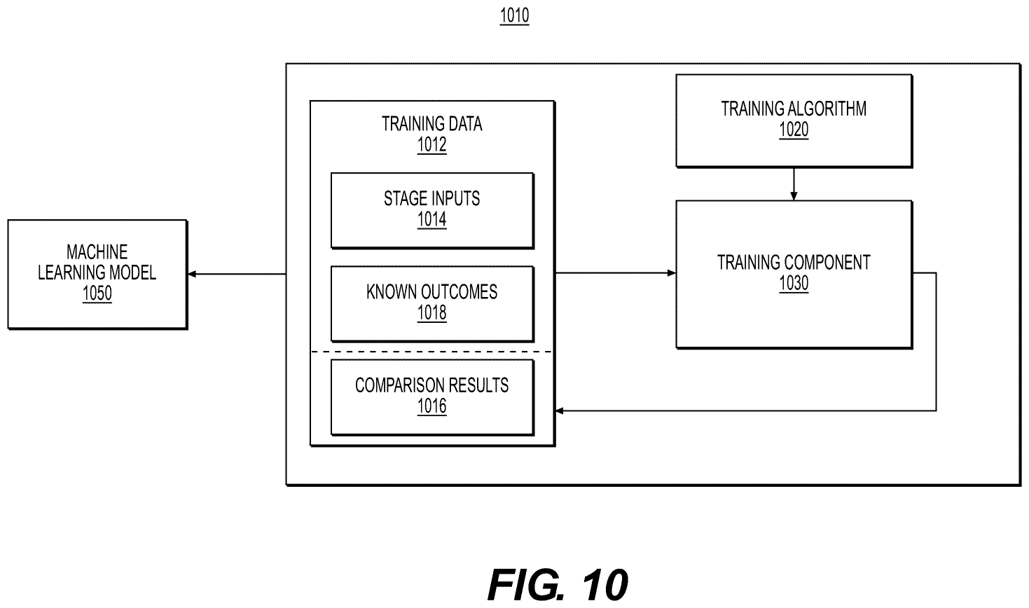

As discussed, one or more implementations disclosed herein may be applied by using a machine learning model. A machine learning model as disclosed herein may be trained using the systems, components, techniques, or the like associated with FIGS. 1-9 disclosed herein. As shown in flow diagram 1010 of FIG. 10, training data 1012 may include one or more of stage inputs 1014 and known outcomes 1018 related to a machine learning model to be trained. The stage inputs 1014 may be from any applicable source including a component or set shown in FIGS. 1-9. The known outcomes 1018 may be included for machine learning models generated based on supervised or semi-supervised training. An unsupervised machine learning model might not be trained using known outcomes 1018. Known outcomes 1018 may include known or desired outputs for future inputs similar to or in the same category as stage inputs 1014 that do not have corresponding known outputs.

The training data 1012 and a training algorithm 1020 may be provided to a training component 1030 that may apply the training data 1012 to the training algorithm 1020 to generate a trained machine learning model 1050. According to an implementation, the training component 1030 may be provided comparison results 1016 that compare a previous output of the corresponding machine learning model to apply the previous result to re-train the machine learning model. The comparison results 1016 may be used by the training component 1030 to update the corresponding machine learning model. The training algorithm 1020 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of the flow diagram 1010 may be a trained machine learning model 1050.

In general, any process or operation discussed in this disclosure that is understood to be computer-implementable, such as those discussed in reference to FIGS. 1-10, may be performed by one or more processors of a computer system. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

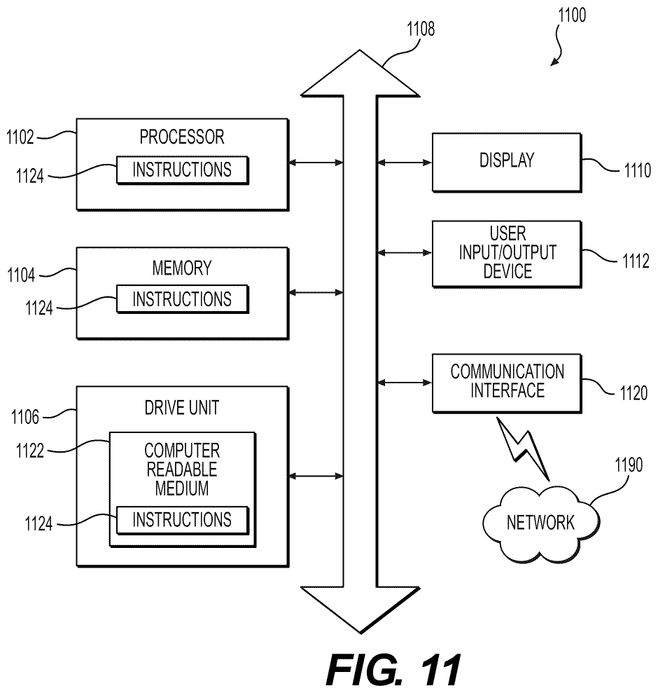

According to implementations disclosed herein, means for collecting, storing, and/or transmitting drug delivery data may be implemented using one or more processors of a computer system, as discussed herein. Drug delivery data may include any data described herein including, but not limited to, data described in reference to FIGS. 1-10, data associated with one or more of bolus amounts, bolus times, basal doses, basal times, chemical stimulation properties (e.g., time, amount, frequency, etc.), electrical stimulation properties (e.g., time, amount, frequency, etc.), and/or the like or a combination thereof. Such means may include collecting, storing, and/or transmitting drug delivery data via wired or wireless communication and may include collecting, storing, and/or transmitting such data including servers, databases, memory, cloud components, and/or components disclosed in reference to FIG. 11, further discussed herein.

A computer system, such as a system or device implementing a process or operation in the examples above, may include one or more computing devices, such as one or more of the systems or devices disclosed in or disclosed in relation to FIGS. 1-10. One or more processors of a computer system may be included in a single computing device or distributed among a plurality of computing devices. A memory of the computer system may include the respective memory of each computing device of the plurality of computing devices.

FIG. 11 is a simplified functional block diagram of a computer 1100 that may be configured as a device for executing the systems and/or techniques of FIGS. 1-10, according to exemplary embodiments of the present disclosure. For example, the computer 1100 may be configured as a system according to exemplary embodiments of this disclosure. In various embodiments, any of the systems herein may be a computer 1100 including, for example, a data communication interface 1120 for packet data communication. The computer 1100 also may include a central processing unit (“CPU”) 1102, in the form of one or more processors, for executing program instructions. The computer 1100 may include an internal communication bus 1108, and a storage unit 1106 (such as ROM, HDD, SDD, etc.) that may store data on a computer readable medium 1122, although the computer 1100 may receive programming and data via network communications. The computer 1100 may also have a memory 1104 (such as RAM) storing instructions 1124 for executing techniques presented herein, although the instructions 1124 may be stored temporarily or permanently within other modules of computer 1100 (e.g., processor 1102 and/or computer readable medium 1122). The computer 1100 also may include input and output ports 1112 and/or a display 1110 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks 1190. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

The subject matter disclosed herein is directed to, for example, the following embodiments:

1. An infusion lead assembly comprising: a housing comprising a needle receptacle, a housing lumen, a pin receptacle, and a housing conductive trace; a connector comprising an connector needle, an internal lumen, a metal pin, and a connector conductive trace; and an infusion lead body comprising an infusion lumen, an exit port, an internal wire, and a distal electrode, wherein the infusion lead assembly forms an electrical path to transmit electrical signals across the connector conductive trace, the metal pin, the housing conductive trace, the internal wire, and the distal electrode and wherein the infusion lead assembly forms a fluid path to transmit fluid across the internal lumen, the connector needle, the infusion lumen, and the exit port.

2. The infusion lead assembly of embodiment 1, wherein the housing further comprises a first gasket forming a fluid seal with the needle receptacle and a second gasket forming a fluid seal with the pin receptacle.

3. The infusion lead assembly of embodiment 2, wherein at least one of the first gasket or the second gasket comprises an in-line filter.

4. The infusion lead assembly of embodiment 3, wherein the in-line filter comprises an anti-bacterial material or an anti-bacterial coating.

5. The infusion lead assembly of embodiment 1, wherein the housing further comprises a housing connection component and the connector further comprises a connector connection component, the housing connection component shaped to receive the connector connection component.

6. The infusion lead assembly of embodiment 1, wherein the housing further comprises a housing connection component and the connector further comprises a connector connection component, the connector connection component shaped to receive the housing connection component.

7. The infusion lead assembly of embodiment 1, wherein the housing lumen is configured to provide fluid communication with the infusion lumen.

8. The infusion lead assembly of embodiment 1, wherein the housing lumen is configured to provide fluid communication with the infusion lumen and the exit port.

9. The infusion lead assembly of embodiment 1, wherein the housing lumen is configured to provide fluid communication with a drug pump, the connector needle in connection with the drug pump via an infusion tube, the infusion lumen, and the exit port.

10. The infusion lead assembly of embodiment 1, wherein the pin receptacle comprises an electrical surface for electrical communication with the internal wire.

11. The infusion lead assembly of embodiment 1, wherein the pin receptacle comprises an electrical surface for electrical communication with the internal wire and the distal electrode.

12. The infusion lead assembly of embodiment 1, wherein the pin receptacle comprises an electrical surface for electrical communication with a pulse generator, the housing conductive trace, the internal wire, and the distal electrode.

13. The infusion lead assembly of embodiment 1, further comprising a proximal electrode.

14. The infusion lead assembly of embodiment 13, wherein the proximal electrode operates as one of a cathode, an anode, or an effective ground.

15. The infusion lead assembly of embodiment 1, further comprising an anchor.

16. The infusion lead assembly of embodiment 15, wherein the anchor is a cuff.

17. The infusion lead assembly of embodiment 16, wherein the anchor is composed of at least one of bioresorbable material or biodegradable material.

18. The infusion lead assembly of embodiment 1, wherein the connector needle is in fluid communication with an infusion tube connected to a fluid pump.

19. The infusion lead assembly of embodiment 1, wherein the connector conductive trace is in electrical communication with a pulse generator cable connected to a pulse generator.

20. The infusion lead assembly of embodiment 1, further comprising an adhesive patch attached to the housing.

21. The infusion lead assembly of embodiment 20, wherein the adhesive patch comprises a securing component to secure the adhesive patch to a user's skin.

22. The infusion lead assembly of embodiment 20, wherein the adhesive patch comprises an open space having an adhesive surface.

23. The infusion lead assembly of embodiment 22, wherein the open space is shaped to receive and secure a portion of the infusion lead body.

24. The infusion lead assembly of embodiment 23, wherein the open space is shaped to allow modification of a length of the infusion lead body that extends from the housing.

25. The infusion lead assembly of embodiment 1, wherein the housing further comprises an infusion gasket.

26. The infusion lead assembly of embodiment 25, wherein the infusion gasket is shaped to receive an insertion needle and provides a fluid seal around the insertion needle.

27. The infusion lead assembly of embodiment 1, wherein the housing is configured to be placed subcutaneously.

28. The infusion lead assembly of embodiment 27, further comprising a subcutaneous injection port.

29. The infusion lead assembly of embodiment 28, further comprising a sealing gasket sealingly attached to the subcutaneous injection port.

30. The infusion lead assembly of embodiment 1, wherein one or more of the infusion lumen, the exit port, the internal wire, or the distal electrode are formed of bioresorbable material or biodegradable material.

31. The infusion lead assembly of embodiment 1, further comprising a receiver configured to wirelessly receive electrical signals.

32. An infusion lead assembly comprising: a receiver comprising a receiver antenna to wirelessly receive power from a transmission component; a subcutaneous housing comprising a needle receptacle, a housing lumen, a pin receptacle, and a housing conductive trace; and an infusion lead body comprising an infusion lumen, an exit port, an internal wire, and a distal electrode, wherein the infusion lead assembly forms an electrical path to transmit electrical signals across the receiver antenna, the housing conductive trace, the internal wire, and the distal electrode and wherein the infusion lead assembly forms a fluid path to transmit fluid across the infusion lumen and the exit port.

33. The infusion lead assembly of embodiment 32, wherein the power is transmitted by a transmission module.

34. The infusion lead assembly of embodiment 32, wherein the subcutaneous housing further comprises a subcutaneous injection port.

35. The infusion lead assembly of embodiment 34, wherein the subcutaneous injection port is in fluid communication with the infusion lumen.

36. The infusion lead assembly of embodiment 32, wherein the receiver antenna includes a coil.

37. The infusion lead assembly of embodiment 32, further comprising an anchor, wherein the anchor is composed of at least one of bioresorbable material or biodegradable material.

38. A method for multimodal stimulation, the method comprising: receiving an electrical signal at a connector; transmitting the electrical signal through a connector conductive trace, a connector metal pin, a housing pin receptacle, a housing conductive trace, and an infusion lead body internal wire to an internal infusion lead body distal electrode at a first time; receiving a fluid at the connector; and transmitting the fluid through a connector internal lumen, a connector needle, a housing needle receptacle, a housing lumen, and an infusion lead body infusion lumen to an infusion lead body exit port at a second time.

39. The method of embodiment 38, wherein the first time and the second time are approximately a same time.

40. The method of embodiment 38, wherein at least one of the electrical signal or the fluid is received based on a user input.

41. The method of embodiment 38, wherein at least one of the electrical signal or the fluid is received based on a pre-programmed setting.

42. The method of embodiment 38, wherein the electrical signal is received at a wireless subcutaneous receiver.

43. The method of embodiment 38, wherein the fluid is received at a subcutaneous injection port.

44. A method for infusion lead body placement, the method comprising: loading an infusion lead assembly onto a needle comprising a needle tip at a first end and a needle hub at a second end opposite the first end, the infusion lead assembly comprising the infusion lead body having a proximal end and a distal end, wherein the loading comprises inserting the needle tip via the proximal end and through the distal end, the distal end comprising a distal electrode and an exit port; inserting the infusion lead assembly loaded onto the needle through a user's skin such that the needle tip is adjacent a nerve and the needle hub is external to the user's skin; applying a force to position the needle hub towards the user's skin; applying a force to the needle hub to advance the infusion lead assembly loaded onto the needle such that the infusion lead assembly loaded onto the needle advances substantially parallel to the nerve; and removing the needle from the infusion lead assembly such that the distal end comprising the distal electrode and the exit port are substantially parallel to the nerve.

45. The method of embodiment 44, wherein the infusion lead assembly is more flexible than the needle.

46. The method of embodiment 44, further comprising placing the proximal end in an open space of an adhesive patch of the infusion lead assembly.

47. The method of embodiment 44, further comprising transmitting an electrical signal through the infusion lead assembly to the distal electrode at a first time.

48. The method of embodiment 47, further comprising causing fluid flow through the infusion lead assembly to the exit port at a second time.

49. The method of embodiment 48, wherein the first time and the second time are a same time.

50. The method of embodiment 44, further comprising: transmitting an electrical signal to the distal electrode; and receiving a response indicating a position of the infusion lead assembly based on transmitting the electrical signal to the distal electrode.

51. The method of embodiment 44, further comprising: providing a detection medium via the exit port; and determining a position of the infusion lead assembly based on detecting the location of the detection medium.

All of the aspects described in the present disclosure (including references incorporated by reference, accompanying claims, abstract and drawings), may be combined in any order, in part or in full, or in any combination or modification, except when such are incompatible or inconsistent. Furthermore, each aspect may be replaced by alternative features serving the same, equivalent or similar purpose, unless expressly stated otherwise or inconsistent with the teachings herein. Thus, unless expressly stated otherwise, each aspect disclosed herein may be only an example of equivalent or similar features. It is intended that the invention be defined by the attached claims and their legal equivalents.

1. An infusion lead assembly comprising:
a housing comprising a needle receptacle, a housing lumen, a pin receptacle, and a housing conductive trace;
a connector comprising an connector needle, an internal lumen, a metal pin, and a connector conductive trace; and
an infusion lead body comprising an infusion lumen, an exit port, an internal wire, and a distal electrode, wherein the infusion lead assembly forms an electrical path to transmit electrical signals across the connector conductive trace, the metal pin, the housing conductive trace, the internal wire, and the distal electrode and wherein the infusion lead assembly forms a fluid path to transmit fluid across the internal lumen, the connector needle, the infusion lumen, and the exit port.

2. The infusion lead assembly of claim 1, wherein the housing further comprises a first gasket forming a fluid seal with the needle receptacle and a second gasket forming a fluid seal with the pin receptacle.

3. The infusion lead assembly of claim 1, wherein the housing further comprises a first gasket forming a fluid seal with the needle receptacle and a second gasket forming a fluid seal with the pin receptacle and wherein at least one of the first gasket or the second gasket comprises an in-line filter.

4. The infusion lead assembly of claim 1, wherein the housing further comprises a first gasket forming a fluid seal with the needle receptacle and a second gasket forming a fluid seal with the pin receptacle, wherein at least one of the first gasket or the second gasket comprises an in-line filter, and wherein the in-line filter comprises an anti-bacterial material or an anti-bacterial coating.

5. The infusion lead assembly of claim 1, wherein the housing further comprises a housing connection component and the connector further comprises a connector connection component, the housing connection component shaped to receive the connector connection component.

6. The infusion lead assembly of claim 1, wherein the housing further comprises a housing connection component and the connector further comprises a connector connection component, the connector connection component shaped to receive the housing connection component.

7. The infusion lead assembly of claim 1, wherein the housing lumen is configured to provide fluid communication with a drug pump, the connector needle in connection with the drug pump via an infusion tube, the infusion lumen, and the exit port.

8. The infusion lead assembly of claim 7, wherein the drug pump comprises a spring system comprising at least one spring, wherein depressing the spring triggers transmission of the fluid across the internal lumen and wherein retraction of the spring causes a second fluid to be retrieved into the drug pump from an external container.

9. The infusion lead assembly of claim 1, wherein the pin receptacle comprises an electrical surface for electrical communication with a pulse generator, the housing conductive trace, the internal wire, and the distal electrode.

10. The infusion lead assembly of claim 1, further comprising a proximal electrode.

11. The infusion lead assembly of claim 1, further comprising an anchor.

12. The infusion lead assembly of claim 1, wherein the connector conductive trace is in electrical communication with a pulse generator cable connected to a pulse generator.

13. The infusion lead assembly of claim 1, wherein the housing is configured to be placed subcutaneously and further comprising:
a subcutaneous injection port; and
a sealing gasket sealingly attached to the subcutaneous injection port.

14. An infusion lead assembly comprising:
a receiver comprising a receiver antenna to wirelessly receive power from a transmission component;
a subcutaneous housing comprising a needle receptacle, a housing lumen, a pin receptacle, and a housing conductive trace; and
an infusion lead body comprising an infusion lumen, an exit port, an internal wire, and a distal electrode, wherein the infusion lead assembly forms an electrical path to transmit electrical signals across the receiver antenna, the housing conductive trace, the internal wire, and the distal electrode and wherein the infusion lead assembly forms a fluid path to transmit fluid across the infusion lumen and the exit port.

15. The infusion lead assembly of claim 14, wherein the power is transmitted by a transmission module.

16. The infusion lead assembly of claim 14, wherein the subcutaneous housing further comprises a subcutaneous injection port in fluid communication with the infusion lumen.

17. A method for multimodal stimulation, the method comprising:
receiving an electrical signal at a connector;
transmitting the electrical signal through a connector conductive trace, a connector metal pin, a housing pin receptacle, a housing conductive trace, and an infusion lead body internal wire to an internal infusion lead body distal electrode at a first time;
receiving a fluid at the connector; and
transmitting the fluid through a connector internal lumen, a connector needle, a housing needle receptacle, a housing lumen, and an infusion lead body infusion lumen to an infusion lead body exit port at a second time.

18. (canceled)

19. The method of claim 17, wherein at least one of the electrical signal or the fluid is received based on at least one of a user input or a pre-programmed setting.

20. (canceled)

21. The method of claim 17, wherein the electrical signal is received at a wireless subcutaneous receiver and wherein the fluid is received at a subcutaneous injection port.

22. The method of claim 17, wherein transmitting the fluid through the exit port at the second time causes at least one of pain management, vasodilation of a tissue, a vein, and/or nerve, or arteriovenous fistula (AVF) maturation.
